(19) United States
(12) Patent Application Publication
(10) Pub. No.: US 2012/0190938 A1
(43) Pub. Date: Jul. 26, 2012

(54) SYSTEM AND METHOD OF DIAGNOSING ACID REFLUX USING INVOLUNTARY REFLEX COUGH TEST

(75) Inventors: W. Robert Addington, Melbourne Beach, FL (US); Stuart P. Miller, Indialantic, FL (US); Robert E. Stephens, Parkville, MO (US)

(73) Assignee: PNEUMOFLEX SYSTEMS, LLC, Melbourne, FL (US)

(21) Appl. No.: 13/354,100

(22) Filed: Jan. 19, 2012

Related U.S. Application Data
(60) Provisional application No. 61/434,464, filed on Jan. 20, 2011.

(51) Int. Cl. A61B 5/03; A61M 16/14; A61B 5/00

(52) U.S. Cl. 600/301

(57) ABSTRACT

A system and method diagnoses acid reflux. A nasogatric/orogastric (Ng/Og) device is inserted through the esophagus and into the stomach of a patient. The Ng/Og device includes a pressure sensor configured to measure intra-abdominal pressure and a plurality of pH sensors positioned along the Ng/Og device. An involuntary reflex cough epoch is induced within the patient. The intra-abdominal pressure and elevation of reflux along the Ng/Og device is measured. A subsequent step determines the functional status of the gastric valve based on the measured intra-abdominal pressure and the elevational reflux along the catheter created by the increased intra-abdominal pressure that occurs during the involuntary reflex cough epoch.

### PRIORITY APPLICATION(S)

This application claims priority to U.S. provisional application Ser. No. 61/434,464, filed Jan. 20, 2011, the disclosure which is hereby incorporated herein by reference in its entirety.

### RELATED APPLICATION(S)

This application is related to commonly assigned and copending patent application Ser. No. 12/878,257, published as 2011/0040157; patent application Ser. No. 12/878,281, published as 2011/0046653; and patent application Ser. No. 12/878,316, published as 2011/0040211, the disclosures which are hereby incorporated by reference in their entirety.

### FIELD OF THE INVENTION

This invention relates to a system and method for diagnosing acid reflux, and more particularly, to a system and method for diagnosing acid reflux during an involuntary reflex cough test examination.

### BACKGROUND OF THE INVENTION

U.S. Patent Publication Nos. 2011/0040157; 2011/0046653; and 2011/0040211, the disclosures which are hereby incorporated by reference in their entirety, disclose improved techniques for evaluating urinary stress incontinence and use of an involuntary reflex cough as a medical diagnostic tool. These publications also disclose a nasal or oral-esophageal-gastric device, e.g., an Ng/Og with an esophageal cuff that acts as a blocking agent in the esophagus to reduce gastric reflux and/or emesis. The esophageal cuff is one advantageous embodiment and an Ng/Og device without the balloon or cuff is also disclosed.

A reflex cough test analysis is also disclosed, together with summary results. A handheld device is used to process the results of testing using the involuntary reflex cough test. Various urinary bladder catheters are also disclosed that may include various indicators. The Ng/Og device is disclosed with the esophageal cuff balloon to reduce or diminish gastric reflux and/or emesis in surgical, neurological and/or trauma patients. It is desirable if further developments can be accomplished regarding use of the iRCT.

### SUMMARY OF THE INVENTION

A system and method diagnoses acid reflux. A nasogatric/orogastric (Ng/Og) device is inserted through the esophagus and into the stomach of a patient. The Ng/Og device includes a pressure sensor configured to measure intra-abdominal pressure and a plurality of pH sensors positioned along the Ng/Og device. An involuntary reflex cough epoch is induced within the patient. The intra-abdominal pressure and elevation of reflux along the Ng/Og device is measured. A subsequent step determines the functional status of the gastric valve based on the measured intra-abdominal pressure and the elevational reflux along the catheter created by the increased intra-abdominal pressure that occurs during the involuntary reflex cough epoch.

In one example, the chemo-irritant can be induced such as by a nebulizer lumen within the Ng/Og device or using a nebulizer. The pressure within the esophagus above the lower esophageal sphincter (LES) can be measured to aid in determining the functional status of the gastric valve.

### BRIEF DESCRIPTION OF THE DRAWINGS

Other objects, features and advantages of the present invention will become apparent from the detailed description of the invention which follows, when considered in light of the accompanying drawings in which:

FIG. 1 is a high-level flowchart illustrating a sequence of steps that can be used for diagnosing acid reflux using the involuntary reflex cough test in accordance with a non-limiting example.

FIG. 2 is a high-level flowchart illustrating a testing algorithm that can be used with the involuntary reflex cough test in conjunction with an urodynamic evaluation.

FIGS. 3-5 are general views of urodynamic catheters that can be used for urodynamic testing in accordance with a non-limiting example, and also showing a urinary incontinence pad in FIG. 5 that can be used with the urodynamic catheters shown in FIGS. 3 and 4.

FIGS. 6A-6E are general environmental views of an oral-esophageal and gastric device or catheter (Ng/Og device) with an esophageal cuff (or balloon) to reduce or diminish gastric reflux and/or emesis in surgical/neurological and/or trauma patients and which can be used with the disclosed system and method in accordance with a non-limiting example.

FIGS. 7A-7G are general views showing another embodiment of the oral-esophageal gastric device (Ng/Og device) similar to that shown in FIGS. 6A-6E but having a nebulizer function, pH sensing function and pressure sensing function, wherein the nebulizer can be used with the system and method in accordance with a non-limiting example.

FIG. 8 is a plan view of another Ng/Og device or a catheter that can assess the severity of acid reflux and compare a response of the involuntary reflex cough test and magnitude and be used with the system and method in accordance with a non-limiting example.

FIG. 9 is a fragmentary plan view of a handheld processing device that can be used in conjunction with the Ng/Og device or other catheters and/or nebulizers.

FIG. 10 is a block diagram showing example components of a handheld processing device such as shown in FIG. 9 that can receive data from the Ng/Og device or other catheters and or nebulizers.

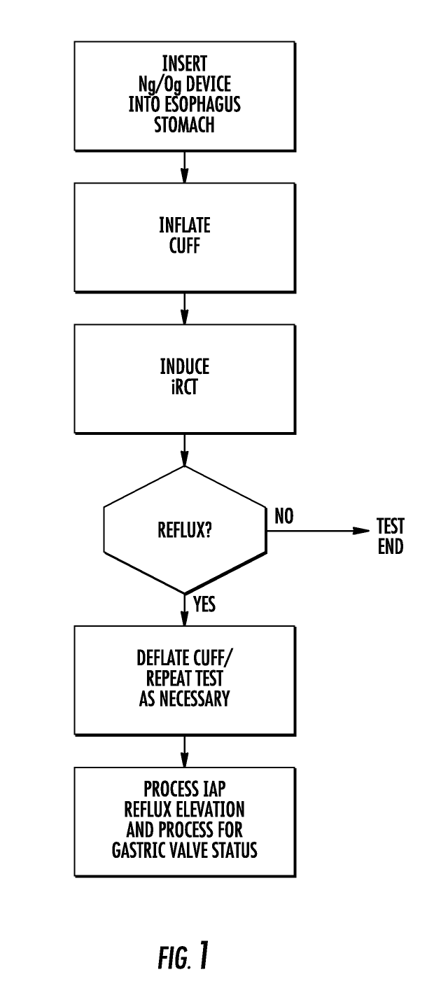

### DETAILED DESCRIPTION OF THE PREFERRED EMBODIMENTS

Different embodiments will now be described more fully hereinafter with reference to the accompanying drawings, in which preferred embodiments are shown. Many different forms can be set forth and described embodiments should not be construed as limited to the embodiments set forth herein. Rather, these embodiments are provided so that this disclosure will be thorough and complete, and will fully convey the scope to those skilled in the art.

The lower esophageal sphincter (LES) does not prevent GERD but assists the gastro-esophageal valve (GEV) with inhalation closure for high pressure. The GEV is the main closure reflux prevention mechanism, not the LES. The iRCT does not allow LES closure. If the GEV is incompetent then the iRCT makes reflux occur for diagnosis. It is believed that the voluntary cough causes LES closure and gives a false negative for identifying reflux severity. The diagnostic features disclosed in the incorporated by reference publications is advantageous for use to aid in diagnosing some reflux issues, but further system and method enhancement is required.

In accordance with a non-limiting example of the present invention, the iRCT can be applied with the system and method, which can determine if a patient has an incompetent gastro-esophageal valve (GEV). This can occur by blocking the lower esophageal closure from the brainstem using iRCT while causing reflux, elevating intra-abdominal pressure and isolating GEV function. The voluntary cough (VC) does not work to provide this function. The use of the iRCT will also aid in diagnosing which patients could benefit from the TIF (transoral incisionless fudoplication) procedure. The use of iRCT in conjunction with the methodology as described below will determine who will benefit from the TIF procedure.

There are known prior art techniques to monitor esophageal pH. In an example, the esophageal pH monitoring can use a thin plastic catheter that is passed through the nostril into the throat and the esophagus as a patient swallows. A sensor on the catheter senses acid and is typically positioned at the catheter tip in a position above the lower esophageal sphincter (LES). A while a recorder is connected to the portion of the catheter protruding from the nose.

The patient carries the catheter and recorder for about 24 hours, typically going about the usual activities of eating, sleeping or working, while any symptoms are recorded by the patient in a diary such as by actuating the recorder. Data is gathered, analyzed and typically graphically portrayed on a display. It is also possible to determine if reflux has entered the upper esophagus or pharynx.

In another example, it is possible to use a capsule to monitor esophageal pH that includes an acid sensing probe, battery and transmitter. Any acid in the esophagus is measured and transmitted to a recorder worn by the patient. Typically, a catheter carries the capsule into the esophagus through the nose or mouth and attaches the capsule to the lining of the esophagus with a clip. The catheter is removed while keeping the capsule in place and the capsule transmits for two or three days until the battery is depleted. The capsule later falls off the esophageal lining about 5 or 7 days later and is passed through the stool. No catheter protrudes from the nose when the capsule is used, and thus, there is greater patient comfort. It is possible for patients to do more of their normal activities and not miss work without feeling self-conscious about the appearance of the catheter protruding through the nose. One disadvantage is that the capsule is not used in the pharynx where it would be uncomfortable and cannot be used in the stomach.

It should be understood that the catheters and Ng/Og device as disclosed in the incorporated by reference '157, '653 and '211 patent publication can be modified for use with the system and method as described below.

In accordance with a non-limiting example, a pH probe as a catheter measures presence of acid reflux and height of acid reflux up the esophagus during an iRCT examination starting above the lower esophageal sphincter (LES). This catheter as a probe ends in the stomach with a pressure sensor that measures intra-abdominal pressure (IAP) during the iRCT for neurological patients. The pH part of the probe is above the LES and the pressure transducer is a pressure sensor in the stomach. In one embodiment, it is possible that the pressure transducer is above the LES and it may work just as well from the esophagus for IAP measurements. The system and method also may decrease the possible false positive events by holding the gastro-esophageal valve (GEV) open in some non-limiting examples of diagnosis. It is possible to use the Ng/Og device as described in the '152, '653 and '211 published patent applications. The iRCT and pH reflux measurements could be performed before opening the reflux blocking balloon. The balloon or other blocking mechanism deflates to allow retesting of reflux or maintains inflation during iRCT testing to protect the esophagus and airway from gastric reflux and from the increased IAP.

In accordance with a non-limiting example, a high-level flowchart is illustrated for the method for diagnosing acid reflux. In one non-limiting example, the Ng/Og device is inserted through the esophagus and into the stomach of a patient. The esophageal cuff is inflated. An involuntary reflex cough epoch is induced within the patient such as delivering the chemo-irritant stimulus via a nebulizer or through a lumen of the Ng/Og tube. Acid reflux is determined by sensing if the first pH sensor that is located at a position above the lower esophageal sphincter and below the esophageal cuff as sensed reflux. The esophageal cuff is deflated and a subsequent involuntary reflex cough epoch is induced within the patient. The intra-abdominal pressure and elevation of reflux along the Ng/Og device is then measured. This information is processed to determine the functional status of the gastric valve based on the measured intra-abdominal pressure and the elevation of reflux along the catheter created by the increased intra-abdominal pressure that occurs during the involuntary reflex cough epoch.

The processing device can be the handheld device shown in FIG. 9 as an example and can test from bladder catheters or Ng/Og esophageal device as described. State of the art reflux diagnostics as described above do not use an involuntary maneuver such as the iRCT. Voluntary cough does not work because even if the GEV is ineffective, the LES closure above it with inhalation gives a false negative. The iRCT inhibits closure of the LES so that the GEV is isolated. The functional status of the GEV can be tested directly in accordance with the system and method as described with elevated IAP to determine if it works properly by visualization during scintigraphy or resultant elevation of reflux captured by a pH probe. It is believed that no one has found a way to isolate the GEV function without the LES interfering and giving false negatives. It is possible to determine risk in neurologically impaired patients and in deciding severity of GERD in normal patients to determine which patient may require a transoral incisionless fundoplication (TIF) procedure.

It should be understood that a catheter Ng/Og device in accordance with non-limiting examples with the system or method can measure from above or below the stomach and measure in some examples from the esophagus. Prior art techniques have used diagnosis techniques with scintigraphy. A patient may drink a contrast and voluntarily cough as a voluntary maneuver. GERD could only be diagnosed with scintigraphy is about 51.2% of patients. The LES blocks and creates problems with analysis.

The involuntary reflex cough test as described blocks the closure of the LES at the brainstem by transmitting signals into the nucleus tract solitarius to the dorsal motor nucleus to the vagus nerve and inhibits the dorsal motor nucleus attended by the nucleus tract solitarius. The patient does not breathe and when that is blocked, what prevents reflux is the GEV. Thus, if it is incompetent, the reflux will shoot up to the hypophraynx or out through the nose. The sensor on the catheter or Ng/Og device can be used in combination to identify pressure either from the stomach or above the stomach. It is desirable not to inhibit the valve or it may give a false positive. Because of the valve, it is sometimes desirable to place the sensor above the stomach and measure pressure at that location and also access how high the pH goes using the same catheter (or Ng/Og device) with a pH sensor or other similar sensor. The catheter or Ng/Og device acts as a probe and plugs into the handheld device and takes pressure and determines airway characteristics and also how high the GERD is in centimeters (cm). The Ng/Og tube as disclosed in the copending applications can extend into the stomach with a pressure sensor, but its size may inhibit closure of the valve where pressure can still be measured and thus care must be exercised.

The probe as a catheter or Ng/Og device typically has a pH or similar sensor that measures pH elevation even when it is not in the stomach and located above the stomach. It is even possible to measure reflux with scintigraphy with the test but that typically is not desirable. It is also possible to determine whether a blocking device or tube (Ng/Og device with a balloon or cuff) is necessary and if a patient has no normal airway protection system and this needs protection. The system and method can also be used to determine if a patient requires the TIF procedure. EMG can also be measured and used as described in the incorporated by reference patent publications.

The catheter or Ng/Og device as a probe, together with the procedure as described, can be used to measure pressure outside the stomach and reflux, and possibly inside the stomach and reflux. It can be attached to an Ng/Og device with the blocking mechanism that measures pressure and reflux.

There is also disclosed an improvement for a single catheter evaluation of urinary function and airway protection using a modified urinary catheter as described in the copending published applications and the processor such as the disclosed handheld device.

Urinary incontinence is prevalent in hospitalized patients or patients in long-term care facilities. Also common among these patients is an impaired airway protection system. Patients with neurologic deficits and/or structural defects involving this system may not be able to safely protect their airway from aspiration. These conditions can post significant risks to the patient by increasing morbidity and mortality. Returning to home from these facilities may not be feasible because of these problems, and as a result, health care costs are significantly increased and patient outcomes can be poor.

It is difficult to evaluate patients for possible bladder dysfunction requiring treatment. It has traditionally required a formal urologic evaluation in a physician's office. Identifying airway protection in neurologically compromised subjects has not been fully established in the healthcare system. Radiologic testing, i.e., modified barium swallow, is difficult to obtain outside the acute care hospital setting and is quite costly.

Formal urologic testing, cystometrogram (CMG), requires the patient to be able to effectively voluntarily cough while a pressure catheter placed in the bladder records the effects of cough on the bladder. Many patients are unable or unwilling to effectively cough in order to obtain useful data during these studies. The use of an inhaled sterile solution of tartaric acid, such as described in the incorporated by reference publications identified above, can illicit involuntary coughs by stimulating the laryngeal reflex cough (LCR). This reflex cough is the patient's first line of airway protection. Because it is an involuntary response, it has shown to be a more reliable test in patients who may have cognitive or neurologic impairments effecting their participation in the voluntary cough testing. With the use of a single urodynamic urinary bladder catheter and the stimulation of the ICR, patients can be evaluated for bladder dysfunction as well as airway protection during one test. This testing may be repeated during the course of their inpatient stay to reevaluate for treatment effectiveness.

Traditional urodynamic testing requires the use of several pieces of equipment located in a dedicated examination room. Portability is very difficult. Patients need to be transported to the physician's office in order to be tested. This is impractical and unfortunately is not available to the numerous patients in need of this testing. Portable single catheter testing can be achieved at the bedside easily and economically. The testing can be done by trained nursing staff, simplifying the process.

The processing device such as the handheld device shown in FIG. 9 captures data from the pressure-time curves obtained during the testing. This information is used to measure and record the area under the curve (AUC), mean intra-abdominal pressure (IAP), peak intra-abdominal pressure, resting detrusor pressure, detrusor voiding pressure, and duration of the cough event. Treatments can be initiated and reassessed for efficacy with repeat testing.

A new testing procedure and methodology for the evaluation is now described.

1) Patient is supine in the hospital bed with HOB (head of bed) elevated 30-45 degrees.

2) Bladder volume is checked a bladder scanner. If the scan results that bladder volume is greater than 100 ml, continue with testing. If the scan indicates bladder volume is less than 100 ml, check the patient again with a bladder scan in approximately 1-2 hours. Once the volume is greater than 100 ml, the testing is continued.

3) Have the patient roll on his/her side, place the HMG electrode to the lumbar region. Place an open adult-sized diaper containing litmus paper under the patient's perineum as the region between the anus and urigenital opening.

4) Prepare the perineum with betadine as an antiseptic or an alternative preparation if the patient is allergic to betadine. The calibrated catheter will be placed using a sterile technique into the urethra. Secure the catheter to the patient's thigh.

5) Record the resting detrusor pressure.

6) Place litmus paper at the perineum near/under the urethra to assess for leakage during the evaluation (or use the urodynamic catheter with sensors).

7) Administer the iRCT.

8) Check the litmus paper for urine leakage.

9) Close the diaper using tabs.

10) Have the patient attempt to void into the diaper.

11) Remove the urethral catheter.

12) Check the bladder scan for PVR (post void residual). Remove the diaper.

It should be understood that “Continence Reflex” is one basis of the Involuntary Maneuver and Stress Incontinence.

It is taught by some that the medical community does not completely understand the support apparatus of the anterior vaginal wall, surrounding muscle and fascial tissues and the neural mechanisms. Some practitioners wonder why urinary incontinence is prevented as a gymnast lands at a high bar dismount.

As addressed throughout this description above and explained partially in the copending publications identified above, inhalation tonicity prevents the incontinence. This “Continence Reflex” holds the smooth muscle IUS (internal urethral sphincter) in place/closed after the deep breath is taken, and then held, during the maneuver. It is believed when the Continence Reflex works, there is deep inspiration, followed by a breath hold and continued closure of the IUS, during gymnastic maneuvers. Striated muscle reflexes are activated as well.

The data contained in the copending and incorporated by reference patent publications identified above support this contention. An issue arises of how to state this pelvic floor/abdomen smooth muscle parasympathetic inhalation activated reflex and its deficits. Differences are present with the striated and smooth muscle reflexes. With iRCT activation, via the Nucleus Tractus Solitarius (NTS), the Respiratory/Inspiratory centers are connected to the inhibition of the Dorsal Motor Nucleus Ten for blocking inhalation, via the Phrenic Nerve, and Vagus Nerve inhibition of the lower esophageal sphincter (LES) smooth muscle contraction while also inhibiting the Periaqueductal Grey (PAG) initiation of the closure of the IUS smooth muscle, during the entire epoch of typically five coughs for the cough event (Average C5 is 14.8 secs). This unguarded pressure over time can identify weaknesses in the striated muscle, somatic nerve innervated, External Urethral Sphincter (EUS) or Intrinsic Sphincter Deficiency (ISD).

Striated muscle reflexes, the anal and EUS, are not inhibited in the brainstem by the Involuntary Cough (iRCT). It is believed that no prior art data has presented this in humans, demonstrating the striated versus smooth muscle reflexes and their differing responses to VC and Involuntary Cough (iRCT). If a patient leaks on voluntary cough (VC) and the iRCT, a clinician may not know where is the level or combination of lesions. It would appear illogical to leak on VC and not leak on the iRCT. It has been found that it did not happen in SUI subjects in some data with a POP-Q of 2 or less. If a patient leaks on VC, there may be a problem with the Continence Reflex. This deficit could occur anywhere along the reflex pathway. Most likely lesion would be below L1 or the Peripheral Nerve. Based on studies, there is typically some age distribution of SUI subjects that leaked on VC and typically they are older. Known diabetics or known peripheral neuropathies were excluded from that study. Spinal Stenosis is often missed and work up seldom done, but it is believed that it accounts for a high percentage of the problem in the lumbosacral spine of older patients, and is affecting peripheral lower motor neurons. It may also involve Onuf's Nucleus at L1 level. Higher lesions may inhibit the continence reflex pathways to Onuf's Nucleus but these are less likely but if it was above L1 involving the spinal cord it would probably be Stenosis as well. Cervical Stenosis is more common than many realize after age 60 and is responsible for a fair amount of radiculopathy. Lesions affecting PAG or above the spinal cord are less likely for causing SUI as the primary complaint, the ability to breathe is higher on the list of problems.

Both VC and iRCT are valuable information along with Urodynamics in determining the mechanisms behind urinary incontinence. iRCT is better than expected, and unmasks early SUI from Intrinsic Sphincter Deficiency (ISD), via EUS (endoscopic ultrasound), in younger patients. The younger subject would not likely have an abnormal POP-Q or an abnormal Continence Reflex mechanism, but could have early ISD from multiple causes, parity, neuropathy, etc. Early identification could lead to helpful noninvasive conservative prevention treatment.

The iRCT is the preferred identification approach to SUI, via sensitivity data, the combination of VC and the IRCT gives more information. It is believed that there are few false positives, regardless of whether the subject has an SUI complaint or not.

The incorporated by reference U.S. Patent Publication Nos. '157, '653 and '211 also disclose urinary catheters that can be modified for use with the evaluation of urinary function and airway protection as described above using a single catheter urinary evaluation as described. The catheters that can be modified are disclosed in FIGS. 31-33 of the published applications and can be used in conjunction with the algorithms shown at FIGS. 23-26 and the handheld device, including the circuits and housing shown at FIGS. 27-30 and 39-42. Various test results that prove the use of the involuntary reflex cough test are also set forth in the published applications together with the physiology of why the involuntary reflex cough test is operative.

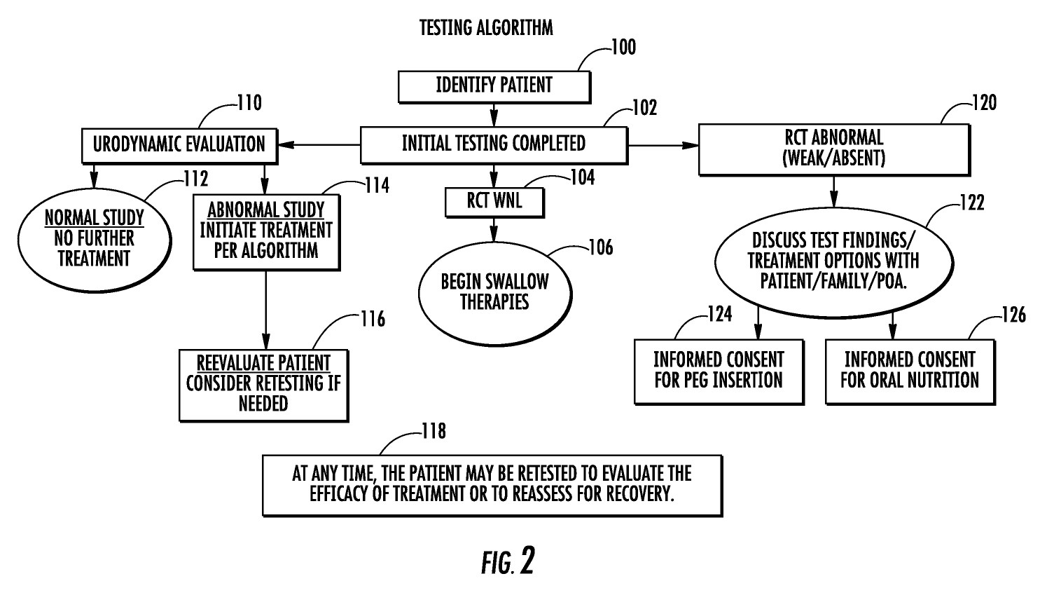

The testing algorithm is illustrated in FIG. 2 and the patient is identified (100). Initial testing is completed (102) and the RCT_WNL is completed (104) followed by swallow therapies (106). A urodynamic evaluation is made (110) and a normal study indicates that no further treatment is necessary 112. If an abnormal study is made, then treatment is initiated per the algorithm 114. The patient can be re-evaluated and retesting considered if necessary 116. It should be understood that at any time, the patient may be retested to evaluate the efficacy of treatment or reassess the recovery 118.

If the reflux cough test is abnormal such as weak or absent 120, the test findings can be discussed with treatment options with the family, the patient, or power of attorney 122. For example there would have to be informed consent PEG (percutaneous endoscopic gastrostony) insertion 124. Also, informed consent would have to be obtained for all nutrition.

There is now described in accordance with a non-limiting example catheter used with urodynamic testing as described above. It should be understood that a single lumen catheter can be used depending on design and needs of one skilled in the art to carry out the system and method in accordance with a non-limiting example. The catheter can be that type of catheter described in the published applications and modified as necessary.

FIG. 3 is an example catheter 1300 that can be used in accordance with a non-limiting example. It is a urodynamic dual lumen catheter formed from a catheter body as an elongated tube with proximal and distal ends and preferably has a smallest external diameter that can contain two lumens within it. It is typically approximately 50 to about 60 centimeters in length. A first lumen 1302 can be used for monitoring bladder activity. In one non-limiting example, it contains a stylet/wire sensor that can be left within the lumen or used alone. A second lumen 1304 permits the filling port to instill fluid into the urinary bladder. The second lumen output is shown at 1306 and a sensor 1308 is positioned at the distal end. This catheter includes a luer lock end for rapid connection to infusion tubing or a syringe, and can accommodate rates of infusion up to 1,200 ml/hr via gravity flow or 15 ml/sec via manual installation.

The external surface of the catheter has a surface area that contains areas of indicators along its length shown generally at 1310 that operate as a urine leak detect device. These indicators 1310 change color when exposed to two components in combination in accordance with a non-limiting example. This color change can occur with a temperature about 30 degrees Celsius and the presence of urea in a non-limiting example.

The catheter 1300 can be used to evaluate bladder pressures at rest, empty, or with urine, filling with fluid during voiding. It is used to evaluate for urinary incontinence by detecting a minimal amount of urine loss during voluntary and involuntary maneuvers of the type as described before. The stylet sensor in one non-limiting example is used alone for pressure monitoring while presenting the least amount of disruption/distortion of the urethra and urinary sphincters. The stylet in another non-limiting example is packaged separately and inserted into an existing Foley catheter to measure pressure and function in one non-limiting example.

In one non-limiting example, the catheter is a dual lumen six French catheter of about 50 centimeters and includes the sensor 1308 and fill port at the second lumen 1304. It is inserted in a non-limiting example about 10 centimeters for a female bladder and 15 centimeters for a male bladder. The location of color change indicators 1310 for a female could be about 11-14 centimeters, and for a male, about 16-19 centimeters. In one non-limiting example, the urine pH range is about 4.6 to about 8.

It should be understood that the catheter is preferably a smaller diameter catheter and includes those catheters of 3 (three) and 4 (four) French. The smallest catheter possible is used as a urethral catheter and somewhat smaller than a standard ten (10) French catheter. It has been found that some patients have a tendency to leak with the larger catheter in place because of the size of the catheter or they become obstructed with that catheter in place. Smaller urinary bladder catheters are typically about 6 (six) French and used for neonatal infants. There are some PICC catheters (Peripherally Inserted Central Catheters) that are three (3) and four (4) French. These smaller catheters should be double lumen in this example. This system is not limited in size, but the smaller is advantageous.

The catheter, in accordance with a non-limiting example as described, can have a first lumen 1302 for a sensor probe 1308 and a second lumen 1304 for the filling with liquid. The sensor probe is a “T-doc” as used with an air-charged catheter for pressure sensing and air-charged pressure recording in one non-limiting example. It should be understood that this catheter can be used with or without filling the bladder, and advantageously used in urodynamic testing. The doctor, nurse or clinician does not have to personally bend down and view the urethra area to determine if there is leakage, which is an advantage in a clinical test. Different types of indicators 1310 as chemical indicators can be used.

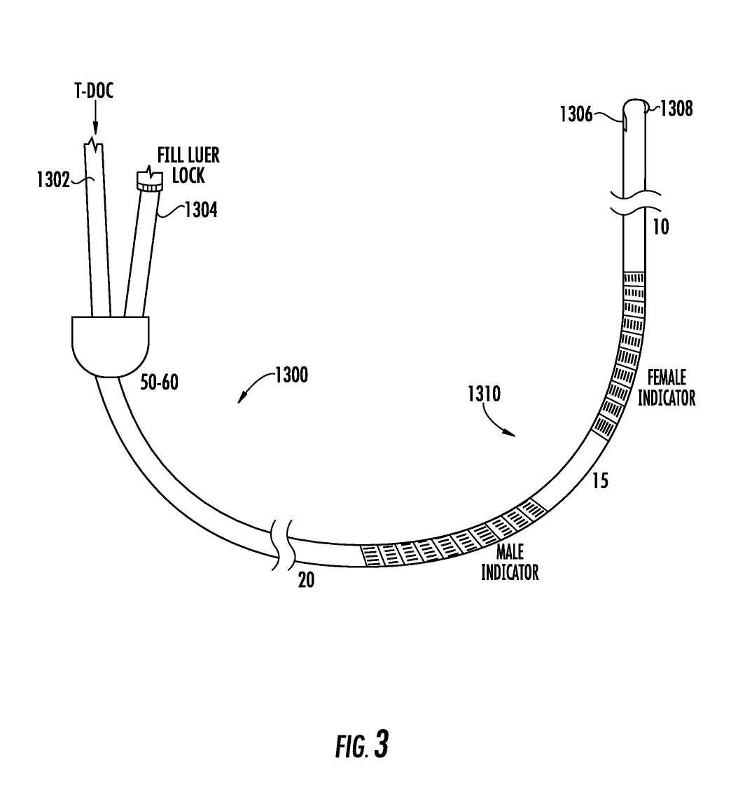

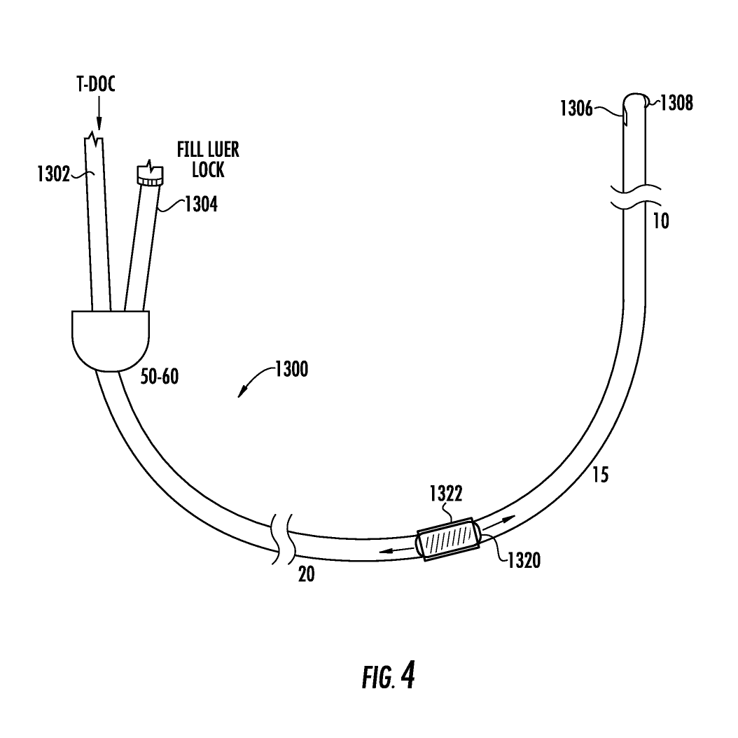

In another non-limiting example such as shown in FIG. 4, the catheter includes a support ring 1320 such as a silastic ring that holds a urine-indicating pad or other enzymatic pad 1322 and is affixed to the catheter as a single unit wherein the catheter that measures the intravascular pressure. The silastic ring 1320 carries a color changing pad in this example instead of using color indicators 1310 positioned along the catheter surface as in the example of FIG. 3. This also provides for a urinary leakage indicator. The support ring 1320 slides on the catheter in one example. It is permanently affixed to the catheter, but adjustable in this example. A moisture indicating dye is used in an example on the pad 1322 positioned on the ring 1320. An example of a dye is disclosed in U.S. Pat. No. 4,327,731 as a moisture indicator, and in one aspect could be an enzyme catalyst.

Different types of pads or substrates could be used in combination with the support ring 1320 and moveable along the catheter. This combination catheter and the urine indicating sensor, in one example, are specific for use to determine an instance of stress urinary incontinence. It is possible, however, to add a balloon to this catheter similar to a Foley catheter such that the catheter remains in place. Two catheters are thus possible. For example, a specific catheter and urine indicator are used for stress urinary incontinence. It is also possible to add a balloon with the larger 14, 16, 18 or 20 French catheters as a larger size. A sensing system is included in this example. Added to this catheter is a channel for urine drainage, the sensor, and an indwelling balloon to keep it in place. The catheter, in one example, is used to determine whether the patient can protect their airway in conjunction with the involuntary reflex cough test (iRCT).

The cloth or pad 1322 is attached to the support ring 1320 and includes on the pad a regent that can be permanently attached. It can be a single use catheter for stress urinary incontinence (SUI) testing. It can be included within a test kit and includes the nebulizer (and the drug) for involuntary reflex cough testing as described before.

In one example, it is possible to have a catheter of about three (3), four (4), or five (5) or somewhat larger French that thread inside a regular Foley catheter with pressure measurement capability. The catheter that goes inside the urethra, such as a seven (7) French catheter, can go inside a Foley catheter. In one example, the balloon is part of the smaller catheter and measures or tests for airway protection in the technique as described before.

An enzymatic moisture detector can be used. Initially, any indicators or pad and ring could be covered before catheter use. When needed, the catheter is uncovered and moved into the proper position against the meatus. A first catheter is used with stress urinary incontinence and testing. Another catheter as a second or larger diameter catheter is balloon specific for reflex cough testing to measure intra-abdominal pressure in determination of airway protection.

In an example, temperature is used with the sensor and changes the sensor as an indicator. It is possible to use the presence of urea for sensing urine. One problem is in bladder testing. The bladder is often filled with saline water or other fluid that is not urine. If the indicator is specific to ammonia or urea, then it would not indicate adequately. Temperature is one advantageous solution and a material that is sensitive to temperature change of about 90 degrees is adequate. The fluid is inserted into the bladder and becomes warmer than room temperature. If there is leakage, it changes the color of the catheter even without the presence of urea.

The tip of the catheter can be placed into the urethra and the outside of the catheter includes the indicator. It changes color if there is leakage whether there is urine inside the bladder or just fill. It could change the color of liquid after it leaks. This could be an assurance against false positives such as would occur with perspiration from the doctor's or nurse's hands. If there is a second testing such as in surgery (and the patient hopefully fixed), a different color could be used. In SUI testing, the liquid is placed in the bladder in one example, but would come out a different color when it reacts with the sensor on the bladder near the meatus. This assures that one is viewing a leakage and not a false positive.

There is a possibility for measuring airway using the port in combination. The catheter can be small enough to go into a side port of a Foley catheter similar to a guide wire. Thus it is possible to take the catheter out if it is obstructing in some way and leave a guide wire. It is possible to remove the catheter and still have a guide wire or small catheter that has a sensor probe on the end. Instead of having a dual channel and having a tube inside a tube where one could do a fill around, it is possible to remove the outside tube that is blocking the urethra. It should be understood that the catheter (depending on size and pathophysiology of a patient) can either block the urethra or hold the urethra open, causing additional leakage. Specific catheter designs as described alleviate these problems. With the larger catheters, the larger catheter size is used to fill and is taken out. The inside tube (catheter) stays. A smaller four (4) French catheter has a dual channel, one for the pressure sensor and the other to fill 1200 millimeters an hour and is adequate to cover different possibilities.

FIG. 5 shows an embodiment of a color changing urinary pad 1204 that can be used with a catheter such as described before. The color changing urinary incontinence pad 1400 is used in conjunction with a catheter 1402 and has a small relief cut-out (hole) 1404 in the middle of the pad where the catheter enters. The pad is placed against the underside near the urethra of a female typically and the catheter enters the urethra and extends through the hole in the center of the urinary incontinence pad for fluid flow and testing purposes. The pad could be taped to the underside in the crotch area. For example, when the involuntary reflex cough test is given and the catheter is inserted through the urethra, the patient is prone to leak urine in some examples. This pad includes concentric rings 1408 around the center catheter cut-out at preferred 10 millimeter intervals for a target area of 50 millimeters. In one non-limiting example, a nitrogen-ammonia (NH3) region is used to identify positively the presence of urine on the pad. The target intervals of 10 millimeters each are used to determine how much leakage and incontinence occurs during, for example, a reflex or involuntary cough test as described before. The different concentric areas have different amounts of reagent in a non-limiting example or different reagents to allow different color changes at the spaced intervals depending on the amount of urine leakage.

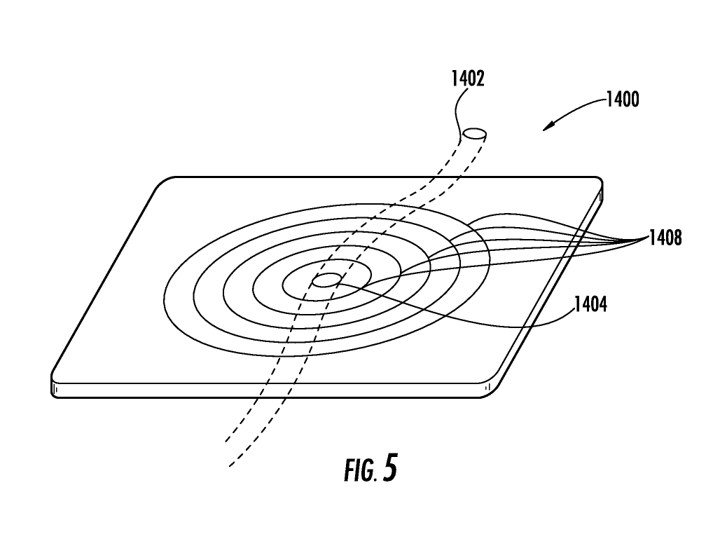

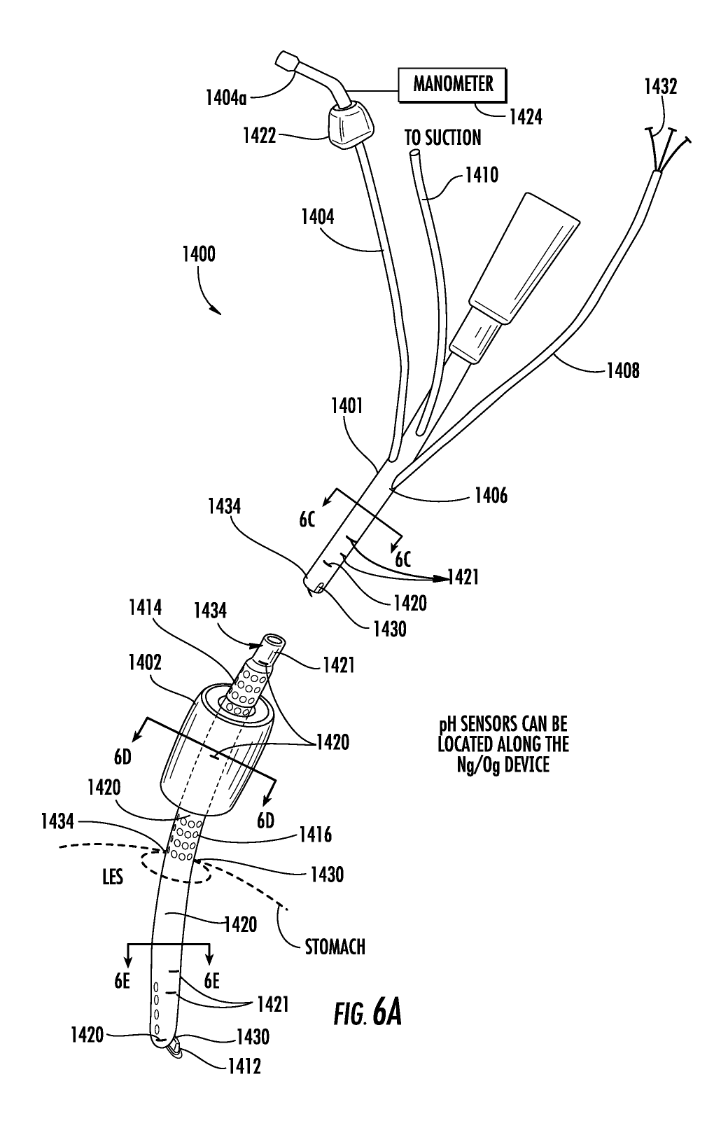

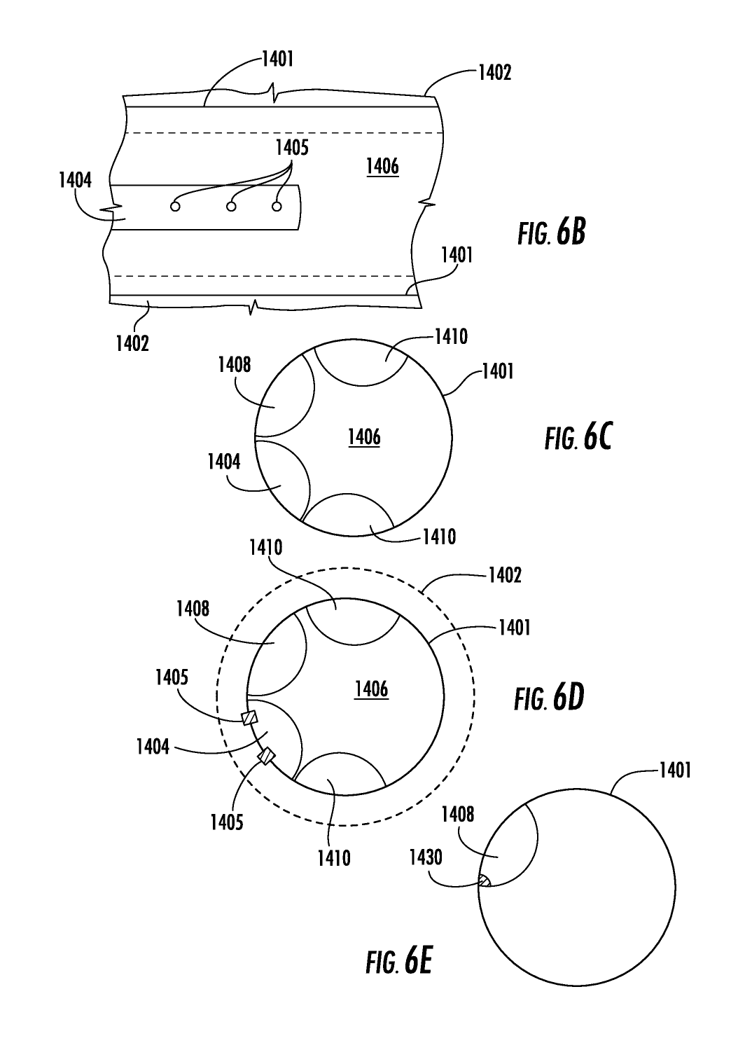

The various Ng/Og devices as disclosed in the published '157, '653 and '211 publications that are incorporated by reference disclose various Ng/Og devices that could be used or modified for use with the system and method in accordance with a non-limiting example. There now follows a description of the Ng/Og devices relative to FIGS. 6A-6E and 7A-7G and FIG. 8 without a balloon. It should be understood that different configurations of the Ng/Og device can be used with the system and method as described to analyze the LES relative to the GEV.

For purposes of description, much of the information regarding possible Ng/Og devices as described in the published patent applications that are incorporated by reference and identified above are set forth.

It is possible to package various components in kit as within a package or housing that includes a nebulizer 1504 for the drug as the tartaric acid in one example and a urinary incontinence pad and an EMG pad to be placed at a paraspinal. A kit could include the nebulizer and various catheters. The various components can be throw away components, except any processing device such as a handheld unit and could include any necessary connector leads that connect into the handheld device or wireless censored device.

Any catheter could include a wireless sensing device that is included in the kit in case wireless technology is used. Although a wireless sensing device could be separately connected to the catheter after the kit is opened, in one aspect, it is possible to include the wireless sensing device connected to any appropriate catheter such that when the kit is open, and the nebulizer removed, the catheter includes the wireless sensing device. The handheld device can be a separate device and the catheter used and wireless signals sent to the handheld device. After analysis and testing on a patient, the kit components such as the catheter and wireless sensing device, pads and nebulizer could be disposed of in the proper manner. It is possible that the EMG pads could connect into the wireless sensing device such that wireless signals are transmitted to the handheld device that includes the pressure readings and the EMG signals. Thus, the kit or system when removed would include the pressure sensing device with the attached leads and EMG pad and catheter that may be integrated together or separately removed and then connected to each other.

FIGS. 6A-6E show an example of the Ng/Og device that can be used or modified. This device could include a foam or air-filled esophageal cuff that is inflated using a separate lumen that is separate from the main lumen and any sump lumen. The device could include a pressure “bubble” at the end of the inflation lumen and could include a manometer connected for measuring pressure, for example, at the esophageal cuff and against the esophageal wall. Another lumen extending through the main body could be included with holes for suction just above the Lower Esophageal Sphincter (LES) to aid in suctioning reflux or emesis. This is advantageous for a surgery patient or acute neural or trauma patient. Details of such device are explained below.

It should be understood that stroke can cause Lower Esophageal Sphincter (LES) weakness. The urology studies discussed above address that determination. The LES is weakened by stroke and other factors, including the initiation of an involuntary cough such as through the iRCT test. The Ng/Og device, in accordance with a non-limiting example and described in detail below, acts as an esophageal reflux protection device to protect the patient from the weakness of the Lower Esophageal Sphincter (LES). It is known that cough causes reflux, which causes more cough. This is a vicious cycle. This device allows blocking of emesis and prevents reflux associated with pneumonia and anesthesia or other functions affecting neural patients. The NG/OG device shown in FIGS. 51a-51e can be used when there is microscopic reflux or massive emesis, which both can cause pneumonia. In some instances, it may be possible to use a Foley catheter and a smaller catheter tube and the Foley catheter left in place and a smaller catheter pulled after cough is measured.

It should be understood that the esophagus is about 25 centimeters long. It is a muscular tube with a diameter of about 2 centimeters average. It tracks the vertebral column curve and descends through the neck and posterior mediastinum and passes through the esophageal hiatus in the right crus of the diaphragm to the left of the median plane at the level of the T10 vertebrae.

The esophagus enters the stomach at the cardial orifice to the left of the midline at the level of the 7th left costal cartilage and T11 vertebra. The abdominal part of the esophagus extends from the esophageal hiatusis in the right crus of the diaphragm to the cardial (cardiac) orifice of the stomach. This area is only about 1.25 cm long.

Food passes through the esophagus rapidly because of the peristaltic action and is typically not dependent on gravity. The esophagus is attached to the margins of the esophageal hiatus in the diaphragm by the phrenicoesophageal ligament, an extension of the inferior diaphragmatic fascia. This ligament permits independent movement of the diaphragm and esophagus during respiration and swallowing. The esophagogastric junction lies to the left of the T11 vertebra on the horizontal plane that passes through the tip of the xiphoid process. Immediately superior to the esophagogastric junction, the diaphragmatic musculature forming the esophageal hiatus functions as a physiological inferior (lower) esophageal sphincter (LES) that contracts and relaxes. The sphincter mechanism for the LES is typically efficient in preventing reflux of gastric contents into the esophagus based on radiological studies. The lumen of the esophagus is normally collapsed superior to this level to prevent food or stomach juices from regurgitating into the esophagus when an individual is not eating.

Barium fluoroscopic studies of the esophagus normally show three constrictions of the esophageal lumen due to impressions from adjacent structures. These are possible locations for placing a device reflux analysis and GERD treatment.

A first constriction is the cervical constriction (upper esophageal sphincter). The superior aspect of the esophagus is the pharyngoesophageal junction, and is approximately 15 cm from the incisor teeth. The cricopharyngeus muscle creates this cervical constriction, which is located at approximately the level of the sixth cervical vertebra.

A second constriction is the thoracic (broncho-aortic) constriction. The arch of the aorta and the left main bronchus cross the esophagus and create esophageal constrictions as seen on anteroposterior and lateral views, respectively. The constriction caused by the arch of the aorta is 22.5 cm from the incisor teeth and the constriction formed by the left main bronchus is 27.5 cm from the incisor teeth.

A third constriction is the diaphragmatic constriction. The esophageal hiatus of the diaphragm is approximately 40 cm from the incisor teeth and forms the diaphragmatic constriction. This is at the level of the lower esophageal sphincter.

The presence of these constrictions is important when placing the device as described with the esophageal cuff, which would help prevent the reflux of gastric contents into the upper esophagus and pharynx. The placement of the device in one example is suggested inferior to the broncho-aortic constriction (27.5 cm from the incisor teeth), but superior to the diaphragmatic constriction at 40 cm from the incisor teeth. The device typically should not be placed in regions of the esophagus with pathological involvement of the esophagus.

FIGS. 6A-6E show the device in plan and sectional views and indicated generally at 1400, and includes a main device body 1401 and a foam or air-filled esophageal cuff 1402 with a separate inflation lumen 1404 for inflation and deflation as shown in FIGS. 6B-6D. FIG. 6B shows the cuff 1402 in deflated position and FIG. 6D shows the cuff inflated. Air channels 1405 connect the inflation lumen and the cuff as shown in FIGS. 6B and 6D. The section view in FIG. 5B shows the termination of the inflation lumen.

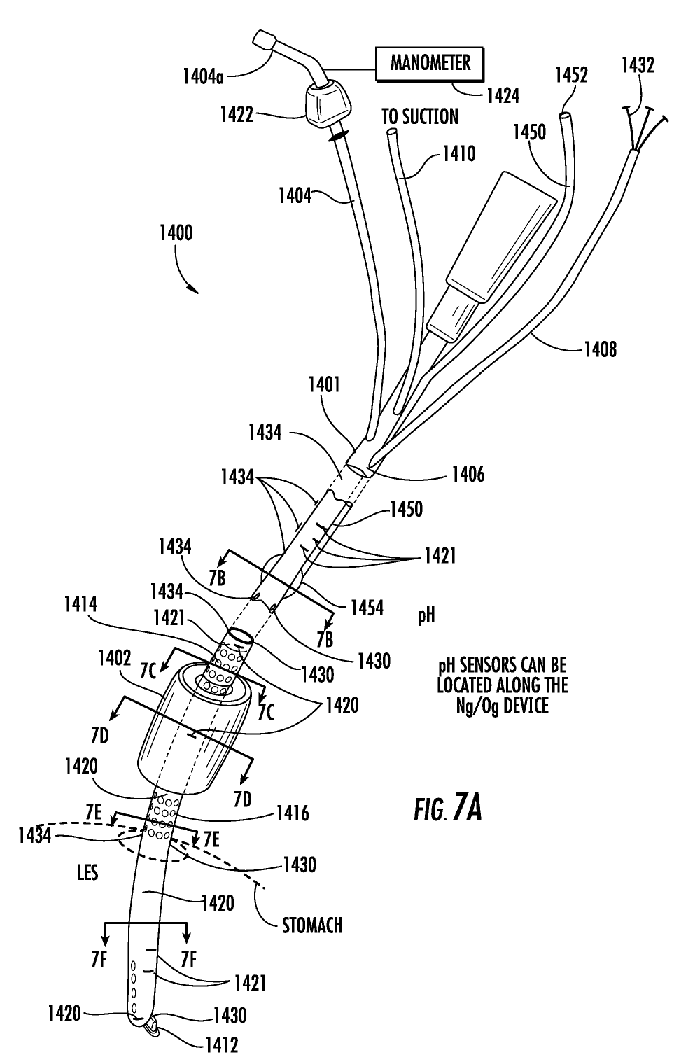

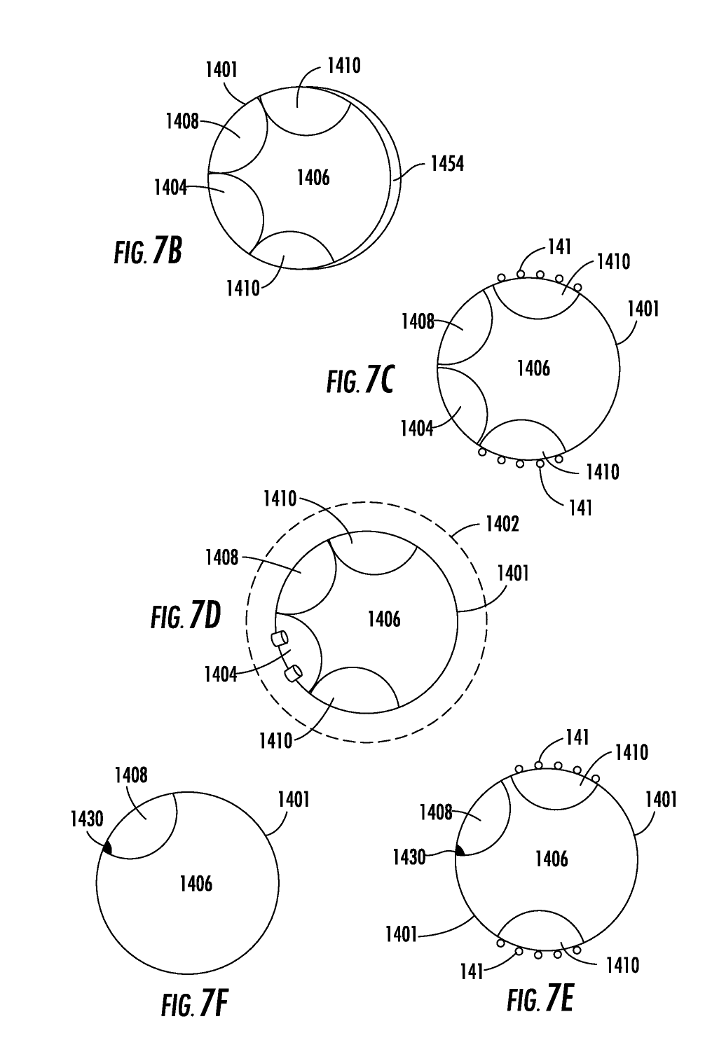

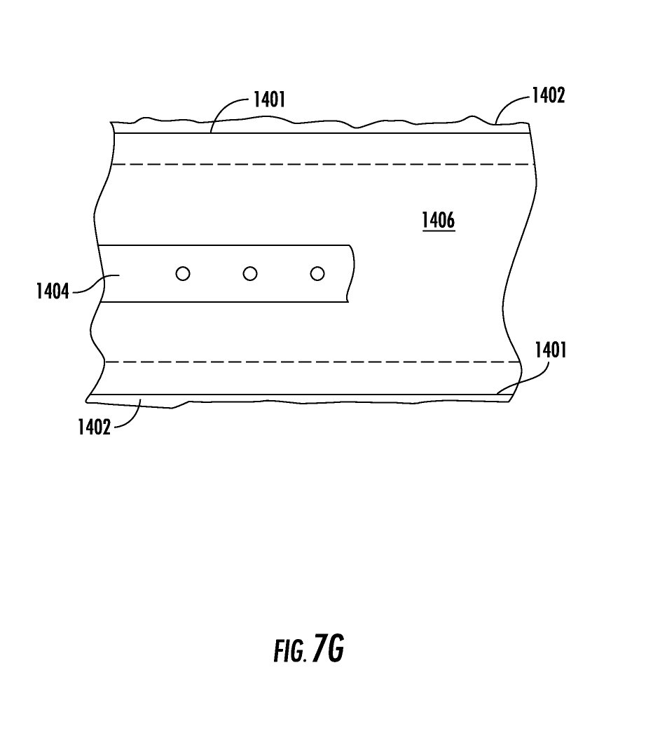

The tip of the device is shown positioned in the stomach, which is shown schematically in FIG. 6A. FIG. 6C is a cross-section taken along line 6C-6C of FIG. 6A. FIG. 6D is a cross-section taken along line 6D-6D of FIG. 6A. FIG. 6E is a cross-section taken along line 6E-GE of FIG. 6A. In these cross-sections, the various lumens are shown, including the main lumen 1406, the sump lumen 1408, the inflation lumen used for inflating the cuff, and any suction lumens 1410 that are used for suction above the LES. The sump lumen 1408 is connected to a sump port 1412 (FIG. 6A) at the end of the device 1400. Drainage holes 1414 positioned in this example above the cuff 1402 allow secretions to pass into the device. These drainage holes could be formed as suction holes such as in the example device described relative to FIGS. 7A-7G and connected to any suction lumens. Suction holes 1416 are positioned below the cuff 1402 and connect to the suction lumens 1410 to permit emesis and reflux to be suctioned. The drainage holes could also connect to the suction lumen 1410 as noted before. In a non-limiting example, the drainage holes and suction holes include one-way valves to allow emesis to enter, but not return.

This device typically forms as a nasogastric or orogastric tube with a Salem sump port 1412 and an additional port 1404a for air entry and exit to and from the esophageal cuff, allowing a high volume and low pressure cuff 1402 as illustrated and supplied by the inflation lumen 1404. The device can come in variable sizes and lengths depending on patient needs and requirements and typically a standard size for use depending on patients. The device can be used for gastric enteral feedings or gastric decompression resulting from the use of the Salem sump port 1412. The device typically includes radio-opaque markings 1420 throughout the length of the tube as illustrated for measurement and placement. Measured markings 1421 as indicia can be positioned in one example along the length of the tube together with a color changing material or pit sensitive material and at the bulb/cuff for measuring emesis, etc.

The cuff 1402 that is shown is in its inflated position in FIG. 6 and is high volume and low pressure and can be inflated with air. It could be foam filled or a combination of both air and foam. Inflation and deflation is through the leur lock port 1404a that includes the pressure inflation balloon 1422 adjacent thereto. The inflation balloon 1422 allows for a tactile cuff and a gross pressure check such as through a manometer 1424 attached thereto. The leur lock port 1404a attaches in one example to a manometer for actual cuff pressure measurement. The cuff 1402 easily collapses for emergency removal or self-extubation without causing damage to surrounding structures of the esophagus, hypopharynx, pharynx, and oral cavity. The cuff is kept inflated below the capillary pressure of the esophageal wall to prevent ischemia that is typically about 7-8 centimeters (cm) water. As indicated before, there are radio-opaque markings 1420 to aid in device placement confirmation. The cuff can be radio-opaque to aid its placement. The upper portion of the esophageal cuff is typically mildly concave to promote secretion to flow towards openings as drainage holes 1414 (or suction holes if formed as such) in the device in this example. An upward force, such as emesis or vomit on the lower portion of the cuff, expands the cuff outwards towards the esophageal wall to control gastric contents from entering the hypopharynx. The inflation/deflation port 1404a can be a different color than the openings for the sump lumen, the suction lumen and the main lumen. The inflation/deflation port 1404a in one example is fitted with the standard leur lock cap and the inflation/deflation port can be labelled with the term “esophageal cuff” to aid practitioners or identifying.

The Ng/Og device is typically inserted through the nasal cavity or through the oral cavity and enters into the stomach. Measurements can be made from the lips or nares to the TMJ (temporomandibular joint) and to about four-finger breadths to sub-xyphoid. When the esophageal cuff 1402 is deflated, a water-soluble lubricant can be applied to the end of the device to aid insertion. This NG/OG device is inserted in a manner similar to an OGT (orogastric tube) or NGT (nasal gastric tube) (NG/OG tube) with the clinician or nurse using the placement radio-opaque markings 1420 to position the device over the lungs and stomach. Once it is in position, it is possible to use auscultate placement by listening to sounds and using an air bolus into the tube and attempt to aspirate gastric contents from the tube. The tube is secured and its placement confirmed by x-ray (using the radio-opaque markings 1420 for help) with the preferred location inferior to the broncho-aortic constriction while superior to the diaphragmatic constriction. The cuff 1402 is inflated through the inflation lumen 1404 and the cuff pressure typically measured with the manometer 1424. The main lumen 1406 as part of the device body 1401 will have low continuous or intermittent suction and may also be used to administer external feedings.

The device 1400 is advantageous for use such as with the neurologically impaired who are at risk for aspiration of gastric contents, including those suffering from a cerebrovascular accident that could be ischemic, thrombotic or hemorrhagic. The device can be advantageously used for non-traumatic brain injury including incephalopathy or intracranial tumor/mass. The device can also be advantageously used when there is traumatic brain injury and general anesthesia, including intra-operative or post-operative, for example, when the patient is neurologically impaired and may not be able to protect their airways. The device is also advantageously used with neurological disorders including Parkinson's Disease, amyotrophic lateral sclerosis and bulbar impairment, myasthenia gravis, and multiple sclerosis. The device is advantageously used with compromised consciousness such as through alcohol intoxication, drug overdose and psychiatric disorders. Indications for use also include gastric decompression because of the use of the sump port and gastric enteral feedings. There are some contraindications for use of the device, including esophageal disruption, esophageal stricture, esophagectomy, esophageal varices, connective tissue disease involving the integrity of the esophagus and cancer of the esophagus.

In accordance with a non-limiting example, the involuntary Reflex Cough Test (iRCT) is used to evaluate the impairment and/or recovery of airway protection. Cuff pressure can also be measured by the manometer 1424. An advantageous pressure for the cuff 1402 is below the esophageal wall capillary pressure. The use of the involuntary reflex cough test is advantageous for people who are neurologically impaired to check to see if they can protect their airway. In this particular device example, pressure sensing is used in conjunction with the device. EMG determination can also be used, as well as pH sensing. Any transceiver inputs for pressure, pH or EMG could input directly into the handheld device. For example, the device could carry pressure sensors as pressure transducers 1430 at various locations on the device to measure pressure when the device is inserted within the esophagus. The transducers 1430 could have transducer leads 1432 that extend through the sump lumen 1408 or be embedded in a wall of the main tube or one of the other lumens. One pressure sensor or transducer 1430 could be in the stomach (such as at the sump lumen), another at the LES, another at mid-esophageal and/or another at the superior esophageal location. It is possible to use an air charged catheter as a pressure sensor with a separate lumen for determining pressure in the stomach, which can be used to determine intra-abdominal pressure. An air charged catheter would require some calibration. Other sensors as non-limiting examples could use fiber optic or other circuit means. The intra-abdominal pressure can be measured but also intra-thoracic pressure. Reflux can be measured by having pH sensors 1434 as inputs along the side with leads also extending through the sump lumen in this example. The handheld device can connect by wired connection or wireless connection to the various pressure, pH and EMG sensors, probes, pads, transducers, etc. It should also be understood that the catheter can be coated with a color changing material, such as for indicating the extent of acid reflux or emesis.

FIG. 6B shows the main device body in an area around the cuff 1402 with the cuff in a deflated position. FIG. 6C shows the different lumens that extend through the device to the cuff area which is shown at FIG. 6D. The lower portion of the device is shown in FIG. 6E showing the main lumen and the sump lumen.

FIGS. 7A-7F disclose an NG/OG device 1400 similar to that shown in FIGS. 6A-6E with similar components that are common between both devices having common reference numerals. In this particular example, however, the device includes a nebulizer lumen 1450 that is extralumenal to the main device body 1401 and provides a nebulizer function using a separate nebulizer port 1452 from the main lumen. This nebulizer port 1452 connects to an oxygen or air source for delivering medication such as for the involuntary reflex cough test at the esopheryngeal area for inhalation into the pulmonary tree or medicine for treating a patient. As illustrated, the nebulizer lumen 1450 terminates at a nebulizer structure or nebulizer/medication delivery mechanism having a built-in venture 1454 to allow delivery of medication for the iRCT around a portion or all the main device body 1401 forming the tube.

FIG. 7B shows a cross-section taken along line 7B-7B and showing the venturi of the nebulizer and the main lumen 1406, deflation/inflation lumen 1404, suction lumen 1410, and sump lumen 1408 that are similar as with the embodiment shown in FIGS. 6A-GE. The two suction lumens 1410 could merge near the proximal portion of the main body or be separate and provide either common suction at the same time above and below the cuff or individually controlled suction. The suction holes or ports as noted before include one-way valves to allow fluid into the suction lumen 1410, but not out. The valves could be formed as cut flaps that extend inward, but not outward to allow ingress, but not egress. This is advantageous such as when emesis extends upward around the tube from the stomach and can pass into the tube to be suctioned, but not passed back out. Also, secretions, if they get past the cuff, will be suctioned by the suction ports that are located above the cuff as illustrated.

The pressure transducers 1430 are located at various points such as at the distal tip at the sump to measure intra-abdominal pressure. A pressure transducer 1430 can be located below the cuff 1430 and above the cuff 1402 with leads extending through the sump lumen 1408 and connected to the handheld device. A pressure transducer 1430 in one example is located at the sump lumen as shown in FIG. 7F. As noted before, it includes pH sensors 1434 along the device that include leads extending through the sump lumen 1408, allowing pH to be measured to detect when emesis is rising from the stomach and the elevation of emesis. The pH sensors 1434 could be located at different locations such as below the cuff and above the cuff and along the main device body 1401. The coating on the device could indicate pH.

This Ng/Og device as illustrated in FIGS. 7A-7F is a multi-purpose Ng/Og device that can be used in a variety of patients who are at risk for aspiration of gastric contents, elevated intra-abdominal and/or intra-esophageal pressures, and/or abnormal airway protection. The device is not limited to the illustrated embodiments, but can be configured with all or any variation in combination of different components to fit the needs of the patient.

The main lumen 1406 extends the entire length of the device and as noted before, the device has radio-opaque markings 1420 along its length, and also measurement markings 1421 as indicia in one example along its length. The entire cuff can be radio-opaque to enhance placement. This device 1400 permits gastric decompression and can be used with a low continuous or a low intermittent suction to remove gastric contents, including liquids and gaseous materials. The device allows enteral feeding that can be administered into the gastric cavity for nutritional support. Any enteral medication administration allows medications to be administered into the gastric cavity.

The sump port 1412 as noted before is intra-lumenal with its own sump lumen 1408 and is integrated the entire length of the device. The sump port opens at the end of the device and when located within the stomach, as when the device is in operation, prevents adherence of the device to the gastric wall and also vents gastric gaseous build-up.

The nebulizer venturi 1454 permits inhalation medication administration. The venturi 1454 is extralumenal and connects to a high-flow oxygen or air source in a non-limiting example. Nebulized medications are delivered through the venturi 1454, typically at the level of the larynx and hypopharynx. The involuntary reflex cough test can therefore be administered efficiently using the device as described.

The cuff or inflation lumen 1404 provides inflation for the esophageal cuff, which as an inflatable cuff is located at the mid-esophagus section and can be inflated and deflated via the leur lock tip balloon 1422 that provides a “feel” for the practitioner to aid in pressure measurements. The pressure of the cuff 1402 can be checked using a manometer 1424, which attaches to the leur lock tip. Gross pressure can be tested manually using the indicator balloon. The esophageal cuff 1402 provides a barrier for any refluxed gastric material from entering the upper esophagus and airway. An unplanned dislodgement of the esophageal cuff does not cause injury because of the particular cuff structure as a flexible material and its configuration to collapse when necessary. Also, the amount of pressure is not excessive enough to harm the esophageal wall in most instances.

The esophageal suction ports 1416, which in this embodiment are both above and below the cuff, permits suction to occur and uses one-way port holes that are located above and below the esophageal cuff such that emesis, reflux and other material can be sucked into the suction lumen 1410 but not pass out. The suction ports 1416 open with the administration of low pressure and intermittent suction. Low suction can be applied to remove the refluxed gastric material in the lower esophagus below the esophageal cuff. The low suction can also be applied to remove material such as, but not limited to, oral or nasal secretions, medications and/or tube feeding material that is collected in the esophagus above the esophageal cuff. For purposes of identification to the nurse or other practitioner, it can be labelled as “Intra-Esophageal Access: Do Not Instill.”

The sump lumen typically will carry transducer leads that extend in the lumen and out past the discharge end of the sump lumen 1408, but the leads could be embedded in the wall of the device. The handheld device or other processing device can connect wirelessly or by wired connection to the transducer leads and monitor pressure within the upper esophagus, the lower esophagus, and within the gastric cavity. Sensors or probes for pH 1434 can be included as noted before and have leads extending through the sump lumen 1408 and out past the proximal end. The leads extending out of the sump lumen for those sensors, transducers or probes can connect to a transceiver for wireless signal transmission to the handheld unit (or wired connection) in one embodiment. Any pressure transducer can send its signal not only into the handheld device, but also into a monitoring system that includes alarms to notify the staff of any increased pressures above or below the esophageal cuff or within the gastric cavity. Sensors for pH can be configured to sound an alarm such as when emesis occurs.

Typically, the nebulizer venturi 1454 will be positioned at the level of the larynx between the nasal pharyngeal area/oral pharyngeal area and allow medication to be administered. The device can be used to measure both intra-abdominal hypertension and reflux. The dimensions of this device are typically not larger than a regular NG/OG tube and not larger than 18 to about 20 French. The sump lumen is much smaller as compared to the main tube, but in this example, large enough to accommodate various leads, which could extend through other lumens. The sump lumen, however, typically remains more clean.

The Ng/Og antireflux/emesis device as described with reference to the preceding description can include suction both above and below the Lower Esophagael Sphincter (LES) as explained above. With placement of the “umbrella” or esophageal cuff close to a predetermined level such as 2-3 cm below the aortic esophageal indentation or “aortic notch,” the inflation with saline or air opens a predetermined cuff shape similar to an hourglass cut in half in one non-limiting example. The bowl shape as identified above as an example collects swallowed secretions and allows passage through both directions for gases. The umbrella would open a limited amount under emesis pressure, and a sensor could flag or alert a monitoring system, triggered by the umbrella or cuff opening while at the same time, automatic suctioning could occur above the LES from the port. The device is also a fully functioning feeding tube for food, liquids or medicine to the stomach and acts as a separate reverse channel, to allow suctioning below the LES in the stomach, and the possibility for constant low-pressure suctioning for reflux above the LES. In a preferred example, the device collapses with pulling even if it is not deflated and pulled by a patient for safety. As noted before, xrays can be used to aid placement of the device in the esophagus. This device can be engineered as necessary for any severe neuro functions and risks for LES weakness or increased LER activity because of dysphagia or reflux, and protect general anesthesia patients after extubation. The device is useful for iRCT testing and protects the patient from neutral created anti-acid medicine stomach content reflux the might get past the ASIC receptors or RAR's (retinoic acid receptors).

The nebulizer lumen 1450 in one example typically extends about half the length of the tube, and in an example is flush with the side of the tube. In FIG. 7A, the device is shown broken in sections for clarity since it is not necessary to show the entire length of the device when only major components are to be illustrated. Nebulized medication enters through one of the ports at the top section of the nebulizer lumen, which terminates at the venturi as illustrated. The medication does not pass into the main tube, but around it, for example, at the level of the larynx in this example. For example, the venturi could be located between the nasal pharyngeal and oral pharyngeal and/or distal. Medication can be administered into that portion of the airway.

The suction lumen includes the one-way valves at the suction ports 1416. Suction can be activated as when emesis occurs and it is brought into the lumen. The main lumen 1406 forming the main device body 1401 provides for food and fluid to pass into the stomach while the other lumens as illustrated provide specific functions and are typically integrated with the main device body.

The esophageal cuff 1402 is located on the outside of the main tube and can be inflated and deflated as noted before. The balloon 1422 is located such that the practitioner can manually feel the pressure of the balloon to exert pressure on the cuff 1402. Manually manipulating the balloon can place pressure on the esophagus via the cuff, and thus, the practitioner can use the feel of the balloon and cuff in this non-limiting example such that the cuff will not cause tissue ischemia.

Suction can occur above and below the esophageal sphincter and suction can occur above and also below the cuff. There are, in some of these examples, one-way valves above and below the cuff that allow emesis or other material to go from outside the device to inside the tube. These one-way valves can be passive and fluid can enter through the one-way valves and be pushed down into the stomach or suctioned up in another example. The device is designed such that emesis cannot come up around the tube. This is important when the patient is unconscious and tube fed, allowing protection of the airway for the patient and protecting the patient from any lower esophageal reflux such as with involuntary events. If a patient inhales, the lower esophageal sphincter closes. If an involuntary event, such as an involuntary reflex cough occurs, and a patient has not inhaled, reflux can occur. This has been shown with a damaged or malfunctioning urethral sphincter or damaged or malfunctioning lower esophageal sphincter.

Guardian reflexes are typically parasympathetic driven. The parasympathetics are cranial and sacral and the sympathetics are cervical, thoracic and lumbar. When a patient inhales, the diaphragm drops and activates the dorsal and causes the lower esophageal sphincter (LES) above the stomach to close. If an event occurs and the internal sphincter does not close, the external sphincter is left alone. This is when patients leaked and why they often have stress incontinence. The involuntary cough happens in about 17 milliseconds and they are not able to inhale. There is no parasympathetic to close the inner sphincter, evident in graphs discussed before. This can be explained because the internal sphincter closes with inhalation. Between every cough, there is an inhalation and the diaphragm drops and the dorsal motor nucleus and para-abductal communicate with the parasympathetics from the cranial and sacral distribution. The device as explained is advantageous because when reflux occurs, and if there is an involuntary cough and reflux, the airway is protected, especially if the patient is unconscious.

When the patient aspirates, a practitioner typically may try to neutralize the stomach contents. The airway will not be able to protect itself because of the neutral pH, and the reflex cough will not activate because the acid receptors are not activated (because of the neutralized stomach). This protective device, in accordance with a non-limiting example, is advantageous to protect the patient.

Normally if the contents are acidic, even if a patient is unconscious and the cough is operative, then the patient would cough material out and this material would not move into the lungs. If the stomach has been neutralized, however, the contents of the stomach may go past the acid receptors and vocal cords and there could be an aspiration syndrome.

In the past, NG/OG tubes were not used with a patient that could not protect their airway. This protective NG/OG device as described, however, in accordance with a non-limiting example, is safely used with a patient that cannot protect their airway and especially useful when administering the iRCT in case reflex occurs. The device can be left in a patient for protection.

The sump port 1412 is integrated into the side of the main tube forming the main device body and exits the base of the tube into the stomach. The sump port vents and prevents adherence of the tube to the wall of the stomach if suctioning occurs, preventing complete vacuum and even collapse of the stomach. A pressure transducer is placed at the sump port (FIG. 7F) for pressure measurements. The various sensors, transducers and probes typically may have leads that extend through the sump lumen and extend outward to plug into the handheld device. The pressure of the stomach can be checked to give a measurement for intra-abdominal pressure and aid in determining intra-abdominal compartment syndrome resulting from excess pressure. This could be a resting pressure. It is preferred, of course, in these scenarios, that pressure not extend above 12 centimeters of water, for example, indicative of intra-abdominal hypertension. Thus, the device as described can be used not only to measure intra-abdominal hypertension syndrome, but also to measure reflex cough. Typically, the reflex cough is activated from the nebulizer venturi 1454 when the various leads 1432 are plugged into the handheld device either by wired or wireless connection. This is as effective in some instances as measuring intra-abdominal pressure from the bladder, but there are some evaluations that occur to reflect that the pressure is sometimes higher from the stomach than from the bladder, which could be a reflection of device position.

Typically, voluntary cough is higher from the stomach than from the bladder. During this process, there could be higher reference numbers for normal between the bladder and the stomach. If there is a rise in normal pressures, then there is possible intra-abdominal hypertension. Typically, the bladder is 12 centimeters of water as a cut-off and the stomach could possibly be 20 centimeters of water, but this value is to be determined with greater testing.

This device prevents reflux from hurting a patient. The pressure transducers 1430 located at the stomach below the cuff and at a point above the cuff are advantageous. If there is pressure build-up below the cuff, it is because the patient typically has vomited and there is now fluid rising and there is possibly esophageal stretch that is placing pressure on the esophagus. It is possible to have a continuous read-out at the handheld unit of the various pressures along the esophagus and in the stomach. It is possible to place alarms on the device, which will activate if there is abnormal high or low pressure. For example, an abnormal high pressure could trigger an alarm and a nurse could assess the patient to see if the patient needs to be suctioned, and whether suction needs to occur above or below the cuff. Also, the nurse could determine if there are intra-abdominal high pressures. It should be understood that the main lumen can be used to feed and the different fluid ports, transducers, sensors and other components as described before are positioned around the main lumen based on the necessary physiology and function required for the device.

The esophageal cuff 1402 is an umbrella-type device such that pressure opens the cuff and blocks emesis. This could be dangerous to the esophagus if proper designs are not used for the cuff. The cuff, i.e., “umbrella,” is designed to readily collapse. If the cuff opens because of emesis or reflux, the opening could trigger a transducer operative with the cuff and activate an alarm. A pressure transducer could be located at that cuff location. If pressure occurs at the cuff by opening the cuff, it will set the alarm off. The cuff in one example could be designed as a static blocking mechanism, and thus, be a static cuff, and in other instances a dynamic cuff. The design is important to ensure that the cuff is not rigid such that it would rupture the esophagus.

The cross-section views in FIGS. 7A and 7E show suction ports above and below the cuff (FIG. 7D). Any deflate/inflate port for the cuff could be just above the cuff with a pressure transducer above and below the cuff in a non-limiting example.

As is understood, the esophagus is a low-pressure system, and the cuff will typically operate as a low-pressure system. Low intermittent pressure is about 80 millimeters of mercury, and low continuous pressure is below about BO millimeters of mercury. The esophagus is much smaller and the suction will typically be reduced to ensure that there is no excess pressure against the walls of the esophagus to cause damage. The pressure transducers, if strategically placed depending on the type of patient, can aid this determination. Air charged catheter technology can be used for pressure measurement where changes in physiological pressure are transmitted through a micro-volume of trapped air.

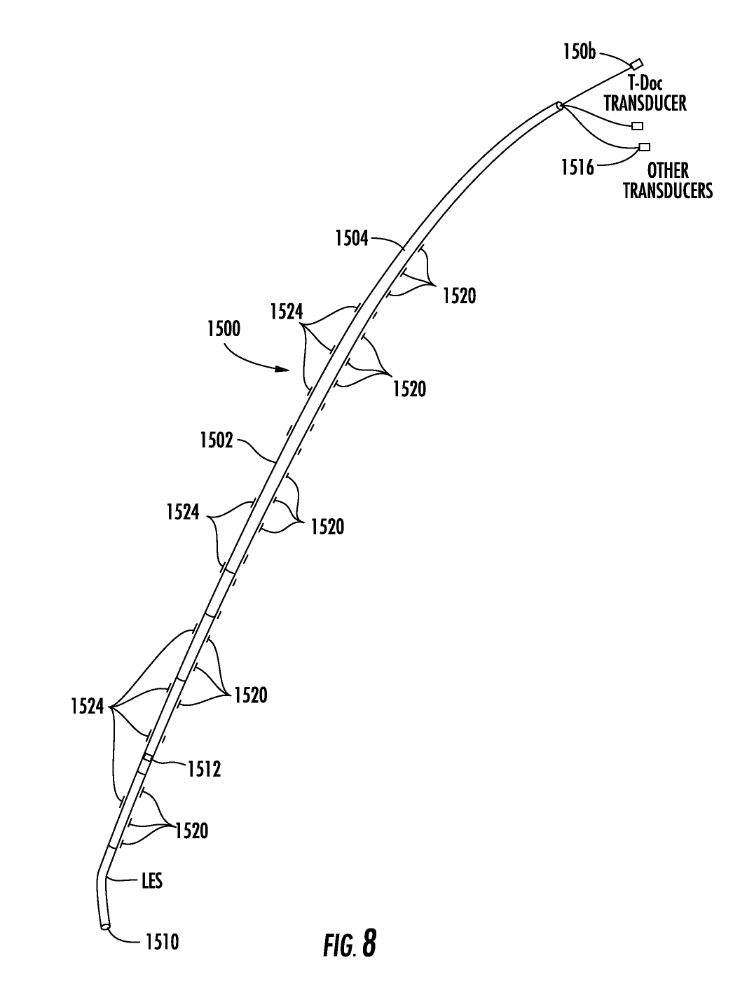

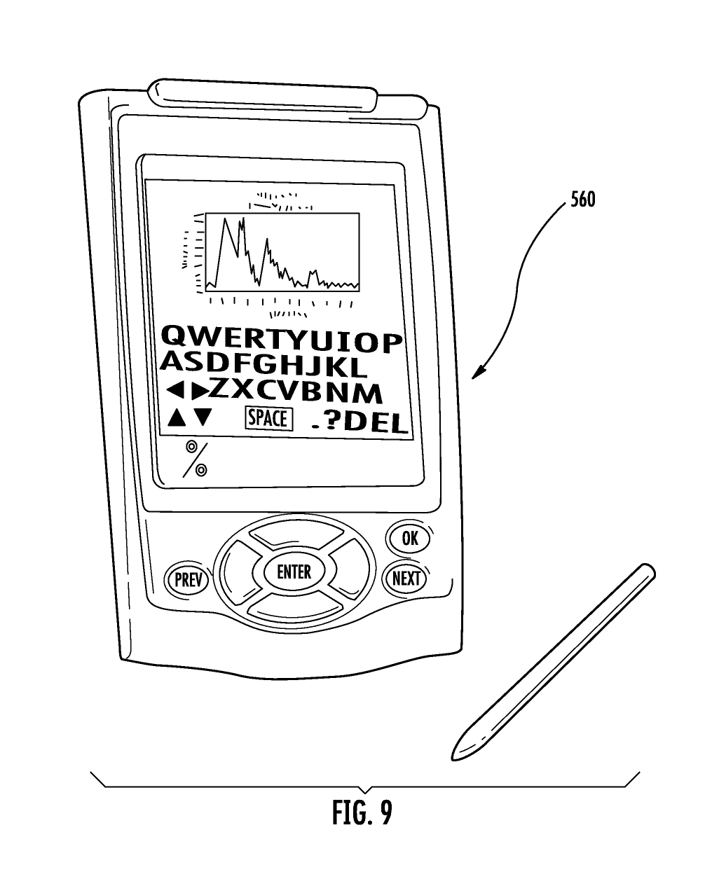

FIG. 8 shows a catheter 1500 as a device used in a method for diagnosing reflux during an involuntary event such as the involuntary reflex cough test. As illustrated, this catheter 1500 does not include any cuff as in previous embodiments shown in FIGS. 6A-6E and 7A-7G and includes a catheter body 1502 having a single lumen 1504 in this example with a T-DOC transducer 1506. It is formed as a small, semi-soft catheter. The adult size is about 6 French and the pediatric size is about 1-2 French. Two pressure sensor areas 1510, 1512 are formed for sensing pressure, for example, by using pressure transducers that are placed at the tip of the catheter and approximately 10-15 centimeters from the tip. Different types of sensors could be used and transducer leads 1516 could extend along the side or in the catheter to the end. The catheter could be an air charged catheter. In one example, the catheter is coated with a pH sensitive material 1520 that will change color when exposed to a pH less than about 4.0, indicating reflux. Measurement markings 1522 can be inserted or printed throughout the length of the catheter. In one example, the catheter is an air-charged (T-DOC) for pressure measurement, but other types of sensing mechanisms such as pressure sensors could be used as understood by those skilled in the art. Fiber optics could be used. The catheter is radio-opaque and includes such markings 1524, if radiologic placement is required and it can include in-patient and out-patient indications.

The catheter can operate as an Ng/Og device and is inserted orally or nasally into the esophagus and through the lower esophageal sphincter (LES) into the proximal stomach. Placement is measured from the lips (oral) or nares (nasal) to the TMJ (temporomandibular joint) to about four-finger breadths sub-xyphoid for adults.

The first sensor 1510 is located in the proximal stomach and can measure intra-gastric/intra-abdominal pressure. The second sensor 1512 is located approximately in the mid-to-lower esophagus and can measure intrathoracic pressure. A pressure grading can be over the LES. EMG information typically can be measured to simultaneously record changes in pressure and gradients. EMG can be measured from the paraspinals as described before. EMG sensors could be located at selected locations on the catheter for EMG measurement in some examples. The catheter can include color change indicia for the pH sensitive material to measure the height of refluxed, acidic gastric contents. The catheter includes pH sensors as noted before.

The catheter 1500 has the potential to identify SUI in conjunction with bladder catheters, assess neurological airway protection (represented as one summated value) and SUI, and additionally assess bladder physiology and categorize any classification with a programmed algorithm in incontinent patients using this one small catheter with EMG measurement. Any inputs of different values can be to the handheld device as described.

When a different type of the same sized air charged gastric catheter is inserted from above, i.e., P.O. or NG, the device will measure neurological airway for protection and assess gastro esophageal reflux from the involuntary maneuver epoch using the iRCT. This gastric catheter, which can also measure pressure below the LES, can predetermine gastric baseline pH and baseline esophageal pH above the LES at standard acid reflux levels already used in other pH testing. From that set up, with any catheters plugged into the handheld device (or eventually wirelessly to a wireless processing device), when given an iRCT, the handheld processing device will assess if reflux is present during the iRCT epoch, such as when it occurs during and/or after the epoch by pH change at these levels. Whether the patients are being treated with acid neutralizers or not, the determined baseline sets the ability to assess pH change when and where in the esophagus it occurs.

This approach will assess the severity of reflux compared to the response of the iRCT and magnitude of the involuntary cough epoch. Depending on the acid reflux elevation compared to the iRCT epoch, without inhalation tonicity protection, it could be instrumental in stratifying reflux severity and pivotal in directing treatment and demonstrating, with repeat testing, the efficacy of the treatment given. This device and process can be used for adult, pediatrics and newborn patients.

If gastroesophageal reflux occurs regularly, it is most likely secondary to an event that is a non-voluntary event, for example, a belch or involuntary cough, thus occurring without inhalation tonicity protection. The reflex acid stimulation to the lung could be from the distal esophagus reflex and very slow causing delayed cough, possibly involuntary coughs (possibly a vicious cycle) or irritable lung reactions causing inhalation and voluntary coughs. Regardless, they would not be temporally correlated by cough and reflux. This is reported in Chang, “An objective study of acid reflux and cough in children using an ambulatory pHmetry—cough logger” published online on Jun. 1, 2010 at Arch Dis Child. The cough sensor as described in Chang could not distinguish the different types of cough.

A question arises if an iRCT epoch when measured is temporarily related to reflux from the stomach during the epoch. It does not matter if there is a small distal esophageal reflux, which is supported by Irwin in the Abstract entitled, “The Cough Reflex and Its Relation to Gastroesophageal Reflux,” Am J Med, March 2000, or a huge geyser airway laryngeal reflux that the ENT's describe as causing severe larynx damage over time because of acid burn. Both reflux events can hurt the lungs over time eventually.

Reflux should be diagnosed during the actual involuntary event when there is little or no inhalation tonicity protection. This will lead to appropriate treatment decisions to protect the lungs, i.e., acid suppression versus Fundoplication. The catheter device as described could be used for airway neuro measurement and bladder physiology, as well as mouth to stomach to prove reflux during an involuntary maneuver. In one example, this may require different types of catheters for different setups that all use the one handheld device for processing.

It should be understood that in the embodiments described above, the cuff operates similar to an umbrella. When the force of emesis hits it, the cuff will expand evenly without tearing or hurting the esophagus. The cuff material is typically a soft material. It should also be understood that this is advantageous because stroke could cause lower esophageal weakness and involuntary cough will not allow a patient to have inhalation protection in some instances. The cuff on the device provides such protection. The NG/OG tube as described with the cuff acts as an esophageal reflux protection device to protect a patient from the reflux caused by any weakness of the lower esophageal sphincter from both involuntary cough or muscle weakness from neurological injury or similar problems. When involuntary cough occurs, the stomach typically does not close down. The cough can cause reflux, which causes more cough as a vicious cycle. In some instances, it is possible have a Foley catheter and the smaller catheter tube as shown in FIG. 53 and leave the Foley and pull the catheter after a cough is measured for reflux. It is also possible that the Salem sump as described can be radio-opaque such as with a coating or a strip itself. The sump port itself could be radio-opaque to indicate where the port extends down into the stomach, such as about 6 centimeters in one example. Capillary pressure of the esophageal cuff can be about 7 to about 8 centimeters of water as a safety factor. The tube feeding channel, such as the main tube, would be a separate channel from the suction channel to ensure that the food is not mixed with any emesis.

The devices, catheter and functions as described above are advantageous. If there is an involuntary cough and reflux, a patient can be protected even if they are unconscious. For example, at times the stomach may be neutralized in a clinical setting and the protective device is advantageous to protect a patient from regurgitating their own stomach contents. Normally, when the stomach contents are acidic, and even if a patient is unconscious, if reflux occurs, a patient would normally cough it out and the reflux or emesis would not pass into the lungs. If the contents are neutral, however, they could discharge past the acid receptors and vocal cords, causing aspiration syndrome. The device and methodology therefore would test and prevent reflux damage and protect a patient's airway. The device can both feed and protect the patient.

Another advantageous aspect is that it is possible to accomplish involuntary cough and measure stomach pressure or intra-abdominal pressure during involuntary cough with the device as described. The involuntary maneuver as a diagnostic tool with the device can be used to diagnose reflux. When the device is pulled out of a patient, the configuration of the cuff allows the cuff to collapse.

The devices can be used to measure the cough epoch in conjunction with EMG. It is advantageous to diagnose the cough epoch and also diagnose severity of disease. The devices in conjunction with other measurements can be used to diagnose severity of reflux during the involuntary epoch and determine the best course of treatment. For example, if surgery is required or pelvic floor exercises or other treatment required. It allows a neuro anatomical finding. The devices can be used to measure pressure such as the abdominal pressure and reflux at the same time not only during the time of the reflux, but also determine the height of the reflux for severity.

It should be understood that a pH probe can be located in the stomach, one at the LES, one at the mid-esophageal region, and one at the superior esophageal region or any combination. pH sensors could be formed electrodes. The devices could have color changing indicia as a coating on all or part of the device to aid in measuring pH and reflux. The devices can include pH sensors and pressure sensors, for example, an air charged sensor. Fiber optics can be used as noted before. A device could be used to protect a patient's airway, feed the patient, administer medication, and vacuum or “suck up” contents and prevent aspiration in the stomach and esophagus. The device operates as a diagnostic tool in another example. The EMC shows a duration of the epoch or event and can be measured. It is typically measured from the paraspinals in an example. The device is used to diagnose GERD and prevent reflux in a non-limiting example.

It should be understood that the involuntary maneuver as described before can be used to test for damaged or malfunctioning abdominal-pelvic intrinsic sphincter. When either a physical or chemical substance stimulates receptors in the laryngeal mucosa, cough may result. Whether the cough is an involuntary reflex or a volitional response depends upon the quantity and type of stimulus. The laryngeal expiratory reflex (LER) is an involuntary, brainstem-mediated reflex. The vagus (X) nerve in one example mediates the afferent component of the LER, and the efferent component is conveyed via the vagus, phrenic, intercostal and abdominal nerves. The reflex cough test (RCT) is a cranial nerve examination assessing both the afferent sensory and efferent motor limbs of the laryngeal expiratory reflex. It is believed that the RCT is presently the only means to test the integrity of the LER.

FIG. 9 is an illustration of an exemplary handheld processing device 560 such as described in the incorporated by reference patent publications. More particularly, it should be understood that this handheld processing device 560 can be used by a nurse practitioner or doctor and receive input as wireless signals or as wired input directly from catheters as Ng/Og devices. Also, this handheld processing device 560 can incorporate the circuit and functions as disclosed in the incorporated by reference publications.

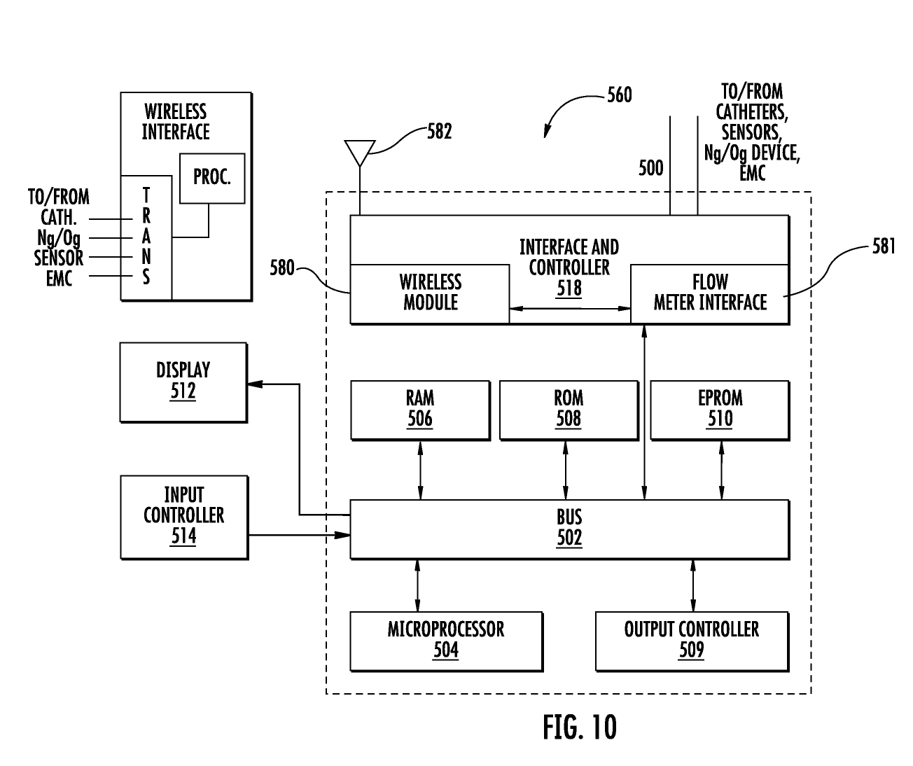

FIG. 10 is a block diagram that illustrates a computer system 500 for the handheld processing device 560. Computer system 500 includes a bus 502 or other communication mechanism for communicating information, and a processor 504 coupled with bus 502 for processing information. Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504.

Computer system 500 may be coupled via bus 502 to a display 512, such as a LCD, or TFT matrix, for displaying information to a computer user. An input device 514, for example buttons and/or keyboard, is coupled to bus 502 for communicating information and command selections to processor 504. Another type of user input device is cursor control, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 operates in response to processor 504 executing one or more sequences of instruction. Execution of the sequences of instructions causes processor 504 to perform the process steps described herein. In alternative embodiments, hard-wired circuitry may be used in place of or in combination with software instructions to implement the invention. Thus, embodiments of the invention are not limited to any specific combination of hardware circuitry and software.

The term “computer-readable medium” as used herein refers to any medium that participates in providing instructions to processor 504 for execution. Such a medium may take many forms, including but not limited to, non-volatile media, volatile media, and transmission media. Non-volatile media includes, for example, optical or magnetic disks. Volatile media includes dynamic memory, such as main memory 506. Transmission media includes coaxial cables, copper wire and fiber optics, including the wires that comprise bus 502. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, a RAM, a PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on a magnetic disk of a remote computer. The remote computer can load the instructions into its dynamic memory and send the instructions over a telephone line using a modem. A modem local to computer system 500 can receive the data on the telephone line and use an infrared transmitter to convert the data to an infrared signal. An infrared detector can receive the data carried in the infrared signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

The handheld device 560 preferably uses wireless technology that could include infrared (IR), Bluetooth, or RFID technology for communicating with the wireless transceiver in the wireless module of the flow meter or part of the nebulizer. The handheld processing device 560 includes a wireless module 580 that would work in conjunction with the transducer interface and controller 518 and the secondary interface 581 and sends and receives readings through the antenna 582 or other system that could be used. The wireless module 580 could be located at different locations.

As illustrated, it is possible to use a wireless interface that interfaces with the handheld processing unit. The wireless interface can include a transceiver and a processor and an interface to catheters, Ng/Og devices, and EMG pads or other similar items. The wireless interface could transmit signals to the wireless unit. It is possible that any catheter body could include a small wireless interface that transmits short range signals to a handheld processing device such as shown in FIG. 9 or a personal computer or similar device.

For purpose of technical instruction, there now follows a general description of physiology for the involuntary reflex cough test (iRCT), which activates the Nucleus Ambiguus, which is also disclosed in some of the incorporated by reference patent publications. The nebulizer with the flow sensing function is adapted for measuring both voluntary cough and involuntary reflex cough, such as explained in the incorporated by reference patent applications. The iRCT selectively activates the Medial Motor Cell Column (MMCC) of the spinal cord rather than the (Lateral) LMCC to fire muscles embryologically predetermined to be involuntary cough activated muscles in the pelvis. In the past, urologists did not selectively activate MMCC without overtly activating the LMCC. Magnetic stimulation or electrical spinal cord stimulation activate both cell columns and thus it is not possible to sort out pathology with these. Magnetic stimulation or other approaches from CNS activation set off both columns.

The pelvic muscles that typically are activated with MMCC cough activation include the lumbar-sacral L5/S1 paraspinal axial musculature, which facilitates inpatient continence screening. An example is through MMCC iRCT muscle activation, obtaining L5/S1 paraspinal firing but not L5/S1 lateral gastrocnemius activation because the gastroc muscles are limb muscles activated primarily through the LMCC.

The L-S paraspinals are easier to access with a large pad placed above the sacrum on the midline that contains active, reference and ground combined. It is not important to determine lateralization of the activity like needle EMG for radiculopathy, but only if activation occurs reflexively where the onset latency is under the pressure activation of the abdomen such as the Levator Ani. This is a poor muscle for these purposes because people train it to activate and set their pelvis if the person senses any intra-abdominal pressure elevation. Also, it is difficult to get pads to stick to that area with hair, perspiration, fungal infections or bowel/bladder incontinence present, and other factors.

Some examples have been developed and studied, including a normal CNS patient with Lumax bladder and bowel catheters and pads at L5/S1 paraspinals and a separate EMG machine and electrodes at the pelvic floor in a standard 3:00 and 9:00 o'clock set-up to demonstrate simultaneous involuntary activation with iRCT. This sets off the pelvic floor muscles.

Thus, normal airway protection data is obtained and normal CNS data to L1 (where spinal cord ends). The set-up includes a complete T12 that cannot void and needs intermittent catheterization with the same set up, thus demonstrating data for normal airway but no L5/S1 EMG activation by MMCC with all the other data necessary to prove an unsafe bladder by the algorithm. A quadriplegic can demonstrate abnormal airway protection and abnormal EMG activation at both paraspinal and pelvic floor muscles with unsafe bladder measurements that follow the algorithm.

It should be understood that iRCT is an involuntary maneuver that activates embryologically predetermined muscles for airway protection and continence that travel primarily through the MMCC in the spinal cord. Different varieties of lesions are captured and determined with summated interval data approach for general screening purposes.

It is known that the laryngeal cough reflex (LCR) is a strong brainstem-mediated reflex that protects the upper airway by preventing aspiration, or the entrance of secretions, food, and/or fluid into the airway below the level of the true vocal cords (rima glottidis), through elicitation of an involuntary cough. The LCR is activated through the stimulation of cough receptors in the vestibule of the larynx. One way this is achieved is through the inhalation of chemostimulants, such as tartaric acid. Studies have shown that if the LCR is intact, the subject will involuntarily cough (normal LCR) upon inhaling a solution containing TA.

In one non-limiting example, the iRCT involves the inhalation of a nebulized 20% normal saline solution of L-TA (Tartaric Acid). Subjects are asked to perform 1 to 3 effective, full inhalations (about 15-20 second exposure by mouth for tidal breathing wearing a nose clip) from a standard jet nebulizer with at least 50 psi from an oxygen wall unit or tank that produces an average droplet diameter of 1 to 2 microns or less. The nebulizer output is 0.58 mL/min. The initiation of an involuntary cough reflex after any one of the inhalations is the end point of the procedure.

Nebulized TA is a chemical tussive that stimulates irritant receptors in the mucosa of the laryngeal aditus. Mild irritation of these receptors results in nerve impulses being conveyed by the internal branch of the superior laryngeal nerve (ibSLN) to bulbar centers of the brainstem. This nerve constitutes the afferent sensory component of the LCR arc. The efferent component of the LCR is mediated through the vagus, phrenic, intercostals and thoracoabdominal nerves.

Inhaled TA is selective in stimulating rapidly adapting (“irritant”) receptors (RARs), in the supraglottic region. In humans, bilateral anesthesia of the ibSLN abolishes TA-induced cough and permits tidal breathing of the nebulized vapor without coughing, supporting the idea that the RARs are responsible for TA-induced cough.

The physiological response from inhalation of TA in a normal subject is abrupt, forceful coughing of short duration. Using a 20% solution of inhaled nebulized TA is a safe, reliable way to assess the sensation in the supraglottic laryngeal region and subsequently the neurologic circuitry of the LCR. In addition, the ability of the iRCT to predict the integrity of the protective LCR in subjects with stroke has been studied.

A 20% solution of TA as an aerosol causes cough by stimulating sensory nerves in and under the laryngeal epithelium. These nerves have been identified histologically, and the reflexes they cause have been identified. The sensory nerves can be stimulated by both non-isosmolar and acid solutions. Tartaric acid may act in both ways, but the balance between them is uncertain.

The nerves are stimulated by the opening of membrane channels in the nerve terminals. More than 20 categories of channels have now been identified, the opening of which will allow calcium flow into the nerve (and also sodium, with exit of potassium), with the result that an action potential is set up, which travels to the brainstem in the central nervous system (CNS), and reflexively induces cough.

Several different types of sensory nerve ending in the larynx have been identified that may mediate cough and other defensive reflexes. They have been extensively studied, mainly in experimental animals by recording the action potentials in their nerve fibers. The probable candidates for cough are the RARs or ‘irritant’ receptors. These are highly sensitive to mechanical stimuli, to hyperosmolar solutions, and to acids.

Once stimulated, the sensory nerves will induce a variety of defensive reflexes, which protect the lungs from invasion of harmful material. These include cough (an inspiration, followed by a forced expiration against a closed glottis, followed by opening of the glottis with an expiratory blast); the laryngeal cough expiratory reflex (LCER, a powerful expiratory effort with the glottis open); and the glottal closure reflex. In some instances a reflex apnea can be produced. The balance of these reflexes may depend on the nature and the strength of the stimulus. In the case of TA, the LCER seems to be dominant, possibly followed by glottal closure, and the pathophysiological advantage of this response in preventing aspiration is obvious.

There now follows an analysis and test results in greater detail that explain the advantageous use of the involuntary reflex cough test (iRCT) for investigating and diagnosing not only SUI, but also physiological abnormalities such as neurologic deficiencies. The nebulizer as described can be used in conjunction with testing. It should be understood that there are differences between normal and neurological patients.

The EMG from the parineal muscles respond almost simultaneously to the onset of the voluntary cough because the patient does not want to leak. With the involuntary reflex cough test, on the other hand, the fast fibers that are set off reach the abdominal muscles quickly, such as in 17 milliseconds as an example. the patient is not able to set their pelvis. In some of the graphs reflecting urodynamic testing as will be described, it is evident that the onset of the EMG activity does not happen at the same time the pressure rises. Some people that have neuropathy, for example, spinal stenosis or nerve injury (even if it is mild), have a situation that prevents the reflexes from closing before the pressure has changed to push on the bladder. It is not possible to obtain this diagnostic tool methodology unless the involuntary cough reflex test is accomplished. When the involuntary reflex cough test is accomplished, it is possible to demonstrate a latency delay and show that the pathophysiology is a neuropathic problem rather than a structural problem. It is possible to separate the pathophysiology using the involuntary reflex cough test and methodology as described.

In one example, a female patient could have a weak spinal cord and her physiology is normal. This patient may not leak during the test, but the patient cannot protect her airway. Thus, using the methodology apparatus and system associated with the involuntary reflex cough test, in accordance with non-limiting examples, it is possible not only to diagnose an unprotected airway, but also to diagnose normal bladder physiology, including the neurophysiology to the patient's sphincter closure process. This is advantageous because it is then possible to determine when someone cannot protect their airway, even though they may have a normal bladder. Conversely, there are patients with a normal airway, but cannot control their bladder. This process and system as described is able to make that diagnosis and thus the involuntary reflex cough test is an advantageous medical diagnostic tool. For example, it is possible to have a patient with a poorly functioning bladder and normal airway and use of the test allows a doctor to find lower urinary tract symptoms and neuropathology. It becomes possible to diagnose a level of lesion in a patient with a full comprehensive neurologic examination using the involuntary reflex cough test, methodology and apparatus as described.

It is possible in one example to measure pressure from a bladder catheter and determine at the same time EMG signals using the EMG electrodes at the L5/S1 in conjunction with the measured involuntary reflex cough test and urology catheter sensing. This is advantageous compared to placing electrodes at the perineal muscles on each side of the sphincter.

It has been found that EMG signals obtained from the perineal muscles have EMG activity from the non-involuntary muscles, i.e., the voluntary muscles blacking out and making analysis difficult because of the signal interference. When the electrodes are placed at the back at the L5/S1 junction, on the other hand, there is nothing else but the paraspinal muscles. It is bone below on each side at the L5/S1 junction. The electrical impulses can be obtained that determine the number of cough impulses coming down through the patient. This is accomplished even if a person has much adipose. The electrode pad used at the L5/S1 junction, in one non-limiting example, typically has an active reference and ground. A pad holds this active reference and ground and the leads as the active reference and ground are plugged into the handheld device (or wireless sensing device in another example) and transmit data to the processor. At least one catheter is also plugged into the handheld device (or wireless sensing device) and measures bladder pressures. A rectal catheter can also be used in some examples. The processor receives EMG signals and determines when the cough event is over.

The involuntary coughs are not hidden by interference when measured from the lower back at the paraspinals as described. This allows a clinician to determine coughs from the bladder when the EMG located at the L5/S1. In one aspect, the area under curve and the average pressure is determined for the cough event corresponding to the involuntary reflex cough test. When this involuntary component of the cough ends, in one example, it becomes silent EMG activity for a period of time. The pressures are at baseline for a period of time, which corresponds in one example to an inhalation. The involuntary component is over.

Sometimes with the involuntary reflex cough test, the cough occurs five times (C5) or even six times without breathing, but when the patient stops to breathe, the event is over. Using the programming applied with the processor in the handheld device, it is possible to calculate the variables inside the wave as to the involuntary cough and determine airway protection capability. Thus, it is possible to determine and measure cough by defining through appropriate data processing the involuntary cough event compared to the whole cough epoch. For example, a patient could cough ten times, but only the first four are part of the involuntary cough event. The coughs after that event are not part of the epoch.

The programming includes algorithm branches resulting in a conclusion of unsafe bladder based on the data analysis. It is possible to calculate from the waveforms information necessary for assessing airway protection ability. It should be understood that taking the EMG from the L5/S1 is also a better situation for the doctor or clinician, and the patient, since it is more acceptable in a hospital, outpatient or inpatient setting. The doctor or clinician does not have to bend down or stoop and look near the crotch area and place pads since the EMG can now be taken from the paraspinals. Also, the placement of pads and electrodes at the paraspinals is advantageous when patients are standing. If pads are placed at the perineal area, sweat and other problems could cause those pads to become loose and good signals may not be obtained. Also, it should be understood that the perineal muscles do not fire involuntarily. The sphincter may fire involuntarily, but that would create more noise as noted before. Electrodes are not placed at the vagina, but are placed at the paraspinal area instead.

This information obtained from iRct and the EMG taken at the paraspinals allows the doctor or clinician to obtain data leading directly to a diagnosis. For example, some patients that have urinary stress incontinence may have a normal airway in this analysis. It has been found by experimentation that the normal airway is about 50 centimeters water average intra-abdominal pressure. It should be understood that the vesicular pressure (bladder pressure) can track intra-abdominal pressure and terms are often similar and used together. “Bladder” or intravesicular pressure is often used to determine and equate with intra-abdominal pressure. The two are sometimes used interchangeably. Stress urinary incontinence and/or bladder physiology can be diagnosed. The system and method as described leads directly to diagnosis. Fifty centimeters average intra-abdominal pressure over time has been found to correspond to an involuntary reflex cough test normal airway. Thus, the standard deviations or other percentages from that value are used in one non-limiting example to determine an abnormal airway. In a conducted study, the actual value is determined to be about 50.6 centimeters water as compared to voluntary cough values of about 48 centimeters of water. In an outpatient setting, it is possible to have the nebulizer (and drug) and only a pad and test SUI. In hospitalized patients or inpatient settings, this combination is used to measure airway and bladder physiology and the test combination includes a catheter.

It should be understood that the involuntary cough reflex test (iRCT) gives a higher pressure average than obtained using a voluntary cough test. The involuntary cough reflex test is thus a valuable medical diagnostic tool. In one example, four variables are significant in this analysis. These variables include: (1) duration of the event; (2) average intra-abdominal pressure of the event; (3) peak intra-abdominal pressure (max) of the event; and (4) area under the curve. Using these four variables, it is possible to process the received data and obtain a specific diagnosis that could not otherwise be obtained without the use of the involuntary reflex cough test. Individual deficits in a specific variable or combination of variables are used to characterize specific diseases and problems and useful as a medical diagnostic tool.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A method for diagnosing acid reflux, comprising:
inserting a nasogastric/orogastric (Ng/Og) device through the esophagus and into the stomach of a patient, the Ng/Og device comprising a pressure sensor configured to measure intra-abdominal pressure and a plurality of pH sensors positioned along the Ng/Og device;
inducing an involuntary reflex cough epoch within the patient;
measuring the intra-abdominal pressure and elevation of reflux along the Ng/Og device; and
determining the functional status of the gastric valve based on the measured intra-abdominal pressure and the elevation of reflux along the catheter created by the increased intra-abdominal pressure that occurs during the involuntary reflex cough epoch.

2. The method according to claim 1, and further comprising introducing a chemo-irritant for inducing the involuntary reflex cough.

3. The method according to claim 1, and further comprising introducing the chemo-irritant through a nebulizer lumen within the Ng/Og device.

4. The method according to claim 1, and further comprising introducing the chemo-irritant by having the patient inhale the irritant using a nebulizer.

5. The method according to claim 1, and further comprising measuring the pressure within the esophagus above the lower esophageal sphincter to aid in determining the functional status of the gastric valve.

6. A method for diagnosing acid reflux, comprising:
inserting a nasogastric/orogastric (Ng/Og) device through the esophagus and into the stomach of a patient, the Ng/Og device a pressure sensor configured to measure intra-abdominal pressure and a plurality of pH sensors positioned along the Ng/Og device including a first pH sensor located at a position above the lower esophageal sphincter and below the esophageal cuff when the Ng/Og device is inserted and an inflatable esophageal cuff;
inflating the esophageal cuff;
inducing an involuntary reflex cough epoch within the patient;
determining if acid reflux has occurred by determining if reflux has occurred at the first pH sensor;
deflating the esophageal cuff;
inducing a subsequent involuntary reflex cough epoch within the patient; and
measuring the intra-abdominal pressure and elevation of reflux along the Ng/Og device; and
determining the functional status of the gastric valve based on the measured intra-abdominal pressure and the elevation of reflux along the catheter created by the increased intra-abdominal pressure that occurs during the involuntary reflex cough epoch.

7. The method according to claim 6, and further comprising introducing a chemo-irritant for inducing the involuntary reflex cough.

8. The method according to claim 6, and further comprising introducing the chemo-irritant through a nebulizer lumen within the Ng/Og device.

9. The method according to claim 6, and further comprising introducing the chemo-irritant by having the patient inhale the irritant using a nebulizer.

10. The method according to claim 6, and further comprising measuring the pressure within the esophagus above the lower esophageal sphincter to aid in determining the functional status of the gastric valve.

11. A system for diagnosing acid reflux, comprising:
An elongate device body forming a nasogastric/orogastric (Ng/Og) device that is configured to be inserted through the esophagus and into the stomach of a patient, the Ng/Og device comprising a pressure sensor configured to measure intra-abdominal pressure within the stomach and a plurality of pH sensors positioned along the Ng/Og device;
a source of chemo-irritant that induces an involuntary reflex cough epoch within the patient; and
a processor configured to receive signals from the pressure sensor indicative of the intra-abdominal pressure and receive pH signals from the pH sensors and process the measured intra-abdominal pressure and elevation of reflux along the Ng/Og device and determine the functional status of the gastric valve based on the measured intra-abdominal pressure and the elevation of reflux along the catheter created by the increased intra-abdominal pressure that occurs during the involuntary reflex cough epoch.

12. The system according to claim 11, and further comprising a nebulizer lumen within the Ng/Og device through which the chemo-irritant is introduced.

13. The system according to claim 11, and further comprising a nebulizer through which the chemo-irritant is introduced.

14. The system according to claim 11, and further comprising a pressure sensor configured to measure the pressure within the esophagus above the lower esophageal sphincter and said processor is configured to receive said pressure measurement to aid in determining the functional status of the gastric valve.

15. The system according to claim 11, wherein the processor is configured to process data from repeated tests to determine the functional status of the gastric valve.
